(19) United States
(12) Patent Application Publication
(10) Pub. No.: US 2012/0064534 A1
(43) Pub. Date: Mar. 15, 2012

(54) APPARATUS FOR PROCESSING A BIOLOGICAL AND/OR CHEMICAL SAMPLE

(76) Inventors: Juergen Pipper, Singapore (SG); Jiri Husak, Singapore (SG); Marek Martinkovic, Singapore (SG)

(21) Appl. No.: 13/255,894

(22) PCT Filed: Mar. 10, 2010
(86) PCT No.: PCT/SG2010/000084

§ 371 Date: Nov. 21, 2011

(30) Foreign Application Priority Data
Mar. 10, 2009 (US) 61158857

(51) Int. Cl. C12Q 1/68; C12M 1/34

(52) U.S. Cl. 435/6.12; 435/287.2

(57) ABSTRACT

The present invention is directed to an apparatus for processing at least one biological and/or chemical sample. The apparatus comprises a substrate, temperature control modules and a rotatable platform carrying a magnetic field generator and an optical unit. The present invention is further directed to a system including the apparatus and magnetically attractable matter as well as method which can be carried out using the apparatus of the present invention.

### CROSS-REFERENCE TO RELATED APPLICATIONS

This application claims the benefit of priority of U.S. provisional application No. 61/158,857, filed Mar. 10, 2009, the content of it being hereby incorporated by reference in its entirety for all purposes.

### FIELD OF THE INVENTION

The present invention refers to an apparatus for processing at least one biological and/or chemical sample, in particular a biological and/or chemical sample in a fluid droplet.

### BACKGROUND OF THE INVENTION

Miniaturization of devices in the chemical, pharmaceutical and biotechnological field has lead to the development of microfluidic devices that control the flow of liquid and permit the performance of a number of chemical and biological reactions. However, such devices do not allow downscaling a conventional, general-purpose chemistry laboratory onto a single microchip due to the lack of appropriate microcomponents, such as microseparators or microfilters or signal detectors. Furthermore, such devices do often not meet mixing requirements. Therefore, an open-well design, typically a multiwell-plate, is frequently employed in combination with automated mixing- and washing devices. Also, such systems perform sample preparation off-chip and rely for example on a conventional microscope for optical measurements, such as fluorescence measurements.

With respect to process miniaturization and automation, the manipulation of droplets has recently received considerable interest due to the possibility of isolating and handling volumes down to the picoliter/femtoliter range (e.g. WO 2004/030820). Several lab-on-a-chip (LOC), micro total analysis (μTAS), and biological microelectromechanical systems (BioMEMS) have been developed for moving, merging/mixing, splitting, and heating of droplets on surfaces, such as electrowetting-on-dielectric (EWOD) (Pollack, M. G. et al., 2000, Appl. Phys. Lett., vol. 77, pp. 1725), surface acoustic waves (SAW) (Wixforth, A. et al., 2002, mstnews, vol. 5, pp. 42), dielectrophoresis (Gascoyne, P. R. C. et al., 2004, Lab-on-a-Chip, vol. 4, pp. 299), and locally asymmetric environments (Daniel, S. et al., 2005, Langmuir, vol. 21, pp. 4240). These methods lack the most important operation for performing sequential biological processes: the ability to separate/purify/isolate starting material and/or reaction products from crude or complex mixtures.

Thus, it is an object of the present invention to provide miniaturized devices which are suitable to overcome at least some of the above problems.

### SUMMARY OF THE INVENTION

In a first aspect, the present invention is directed at an apparatus for processing at least one biological and/or chemical sample. The apparatus comprises or consists of

a substrate having a fluid contact surface; wherein the fluid contact surface comprises a texture and a wettability adapted to allow a fluid droplet arrangeable thereon to remain intact upon being contacted with the fluid contact surface of the substrate;

at least one temperature control module arranged to control the temperature in at least one different temperature zone at the fluid contact surface of the substrate;

a rotatable platform arranged on the opposite side of the fluid contact surface of the substrate; wherein the rotatable platform comprises:

a magnetic field generator; wherein the magnetic field generator is vertically movable parallel to the rotational axis of the rotatable platform; and

at least one optical unit adapted to emit light of at least one specific wavelength and to detect light of at least one specific wavelength.

In another aspect, the present invention is directed to a system comprising or consisting of an apparatus described herein and magnetically attractable matter.

In another aspect, the present invention is directed to a method of processing at least one biological and/or chemical sample. The method comprises or consists of:

disposing at least one fluid droplet onto a fluid contact surface of a substrate of the apparatus described herein; and

performing a process on the biological and/or chemical sample in the at least one fluid droplet; wherein the fluid droplet comprises an inner phase and an outer phase, and wherein the outer phase is immiscible with the inner phase, and the outer phase is surrounding the inner phase, and wherein the inner phase comprises the biological and/or chemical sample, and the inner phase is shielded from the environment by the outer phase; wherein the fluid droplet comprises magnetically attractable material.

In still another aspect the present invention is directed to the use of an apparatus described herein for carrying out a nucleic acid amplification process.

### BRIEF DESCRIPTION OF THE DRAWINGS

The invention will be better understood with reference to the detailed description when considered in conjunction with the non-limiting examples and the accompanying drawings, in which:

FIG. 1 shows a schematic view of the support structure 55 and the rotatable platform 15 mounted on it.

FIG. 2 illustrates a top view of the apparatus with the support structure 55 and the rotatable platform 15 and the fluid contact surface 57 of the substrate.

FIG. 3 shows a sectional view through the substrate, the rotatable platform, an optical unit of the rotatable platform and the support structure.

FIG. 4 shows a diagram illustrating the limit of detection (LOD) which was estimated by diluting 5′-labeled 5′-AGGTCGGGTGGGCGGGTCGTTA-3′ (SEQ ID NO: 4). The solid lines denote the linear regression and the background including 3 times the signal-to-noise ratio (3 SNR).

FIG. 5 shows a diagram with the results of fluorescence measurements generated during a TaqMan based duplex qPCR. Detection of different quantitation standards of the target (open symbols, 0-10⁵ copies/reaction) in the presence of the IAC (solid symbols, 10 copies/reaction). The target [IAC] is labeled by FAM [TxRed] and followed in the blue [yellow] channel. Contrary to an (external) positive control, a nontarget IAC is simultaneously coamplified with the target DNA in the very same fluid droplet. Even though no target DNA is present in the sample, an IAC effects a positive control signal. In this way, a true negative PCR result is distinguishable from a false-negative one due to PCR failure.

FIG. 6 illustrates the standard curve for a HIV-1 qPCR assay. The C_{T} of nine separate experiments (performed in duplicates) were plotted versus the log of the number of HIV-1 cDNA copies. The open black squares are the means of C_{T} values; the solid black line is a linear regression fit (R²=0.9993) of the C_{T} values; the dashed lines denote the upper and lower confidence limits. The PCR efficiency was 91%. 10-10⁹ copies of HIV-1 cDNA were used in 2.5 μL reaction volume.

FIG. 7 depicts non-limiting examples of possible shapes of a temperature control module.

FIG. 8 shows schematically an embodiment of a temperature control module of the apparatus of the invention, seen from below. A heater 600 and a sensor 610 are each concentric, with the heater 600 surrounding the sensor 620. The heat conductor 630 includes two concentric parts, connected by a linker 640, as well as a rod-shaped part of a length 650. In this embodiment, the area of heating zone is defined by the boarders of the heat conductor (630).

FIG. 9 depicts a schematic cross-section of an embodiment of a temperature control module. The sample is a liquid droplet 570, which includes an inner 510 and an outer phase 520. A substrate 530 contacts a concentric heat conductor 540, which is in turn in contact with a concentric heater 550 and a concentric sensor 560.

FIG. 10 depicts a photograph showing the substrate with fluid droplets arranged on the fluid contact surface of the substrate. The temperature control modules are soldered to a Printed Circuit Board (PCB). Situated there above is a transparent square glass slide as a substrate.

FIG. 11 depicts a temperature/time profile during a PCR using the apparatus and method of the present invention. It requires only 2 seconds for a temperature decrease from 94° C. to 54° C., while heating is significantly faster, as it is controlled by a PID system.

FIG. 12 depicts a washing process of a sample in a fluid droplet 700 by means of a second droplet 705 in top view (A) as well as side view (B), depicting a magnet 710 under a fluid contact surface of a substrate 715.

FIG. 13 depicts a genetic analysis of a blood droplet sample using the method of the invention. Leukocytes are bound to functionalized magnetically attractable particles 720 in droplets 735, isolated, washed, thermally lysed by means of thin film heaters 725 controlled by thin film sensors 730, and processed by reverse transcription (RT), followed by polymerase chain reaction (PCR) and pyrosequencing (PSQ). The arrows indicate the direction, in which the sample is moved.

FIG. 14 shows an embodiment of the apparatus of the present invention (FIG. 14(A)). FIG. 14(B) and (C) show a possible combination with a microcontroller unit including a TFT display on a separate STM3210E-evaluation board. The whole assembly shown in FIG. 14(A) weights around 0.4 kg.

FIG. 15 shows the results of magnetic force-distance measurements with a cone shaped permanent magnet made of an alloy of NdFeB (from Neotexx) and a permanent magnet made of two stapled discs.

### DETAILED DESCRIPTION OF THE PRESENT INVENTION

In a first aspect the present invention refers to an apparatus for processing at least one biological and/or chemical sample. The apparatus comprises a substrate having a fluid contact surface. The fluid contact surface comprises a texture and a wettability adapted to allow a fluid droplet arrangeable thereon to remain intact upon being contacted with the fluid contact surface of the substrate. The apparatus also comprises at least one or two temperature control modules arranged to control the temperature in at least one or two different temperature zones at the fluid contact surface of the substrate. Further comprised is a rotatable platform arranged on the opposite side of the fluid contact surface of the substrate. The rotatable platform comprises at least one or two magnetic field generator; wherein the magnetic field generator is vertically movable parallel to the rotational axis of the rotatable platform. The rotatable platform also comprises at least one or two optical units each adapted to emit light of one or at least one specific wavelength and to detect light of one or at least one specific wavelength.

The apparatus described herein can be used for methods for carrying out biochemical or chemical reactions in droplets having a microliter or nanoliter volume. Those methods including small droplets can be carried out at the fluid contact surface of the apparatus of the present invention for example in the manner described in US 20090263870 A1, WO 2007/094744 A1 or Pipper, J., Inoue, M., et al. (2007, Nature Medicine; vol. 13, no. 10, pp. 1259).

Moreover, the apparatus of the present invention allows not only to carry out such biochemical or chemical reactions at the fluid contact surface of the substrate but also to monitor the progress of those reactions using the at least one or two optical units integrated in the apparatus of the present invention. This construction avoids the use of bulky microscopes which in many applications have to be used to either analyze the result of the biochemical or chemical reaction afterwards and separated from the miniaturized device or which have to be placed above the miniaturized device to monitor the progress of the biochemical or chemical reactions while they take place.

In addition, the apparatus of the present invention allows carrying out multiple reactions at its fluid contact surface at the same time. This is of particular importance because measurements of single samples often require control experiments, such as positive or negative controls. The setup of the apparatus of the present invention allows measuring multiple samples at the same time.

Also, the use of one or more optical unit allows subjecting each sample to different wavelength. In case, for example, a polymerase chain reaction is carried out at the fluid contact surface of the apparatus, it is possible to carry out multiplex PCR's including several targets and multiple different optically active substances, such as fluorophores. Thus, the apparatus described herein allows performing sample preparation, processing and monitoring on a small footprint.

In the apparatus described herein the at least one or two optical units which can be used for monitoring the progress of the biochemical or chemical reactions in the biological and/or chemical sample are arranged on or integrated in a rotatable platform. This platform can be of any shape, for example, a square, circular or polyangular, such as hexagonal or octagonal. Rotation of the platform allows positioning the optical units and the magnetic field generator under the droplets at the fluid contact surface of the apparatus. Thus, the magnetic field generator and the at least one or two optical units are arranged on a circular orbit on the rotatable platform. The circular orbit or pathway is aligned with the at least one or two different temperature zones at the fluid contact surface of the substrate. This alignment is desirable to ensure that the droplets can be positioned within the confines of a temperature zone in which the samples to be examined can be subjected to a thermal treatment, such as a thermal cycling required during a PCR. It is also possible to position different optical units and the magnetic field generator on different circular orbits on the rotatable platform. In one embodiment, it is possible to use also more than one magnetic field generator. The different magnetic field generators and optical units can be placed on the same or different circular orbits on the platform. This, way the space at the fluid contact surface of the substrate can be used more efficiently which would also allow increasing the number of samples to be analyzed at the fluid contact surface of the platform. For example, one set of at least one or two optical units and at least one or two magnetic field generator can be positioned on an outer circular orbit of the platform which is closer to the outer boarder of the platform while another set of at least one or two optical units and at least one or two magnetic field generators are positioned on an inner circular orbit of the platform. The position of the optical units on the outer and inner circular orbit relative to each other can be staggered to allow incorporation of several optical units in the rotatable platform.

The function of the at least one or two optical units is two-fold. At first an optical unit is used for excitation of the optical active molecules which can be comprised in the fluid droplets at the fluid contact surface of the substrate. Secondly, the optical unit directs any optical signal generated by any such optical active molecule to a detector. Since some optical active molecules require a certain excitation wavelength, the optical units can be chosen to direct light of different wavelengths to the fluid droplet(s) located on the fluid contact surface of the substrate.

Therefore, an optical unit referred to herein comprises an excitation light source adapted to provide excitation light either of a specific single wavelength or of different specific wavelengths. The optical unit further comprises a detection module adapted to direct light of a specific wavelength or light of at least two specific wavelength emitted by a composition(s) or an optical active molecule(s) comprised in a fluid droplet arrangeable on the fluid contact surface of the substrate to a photo detector. The optical unit(s) further comprise a light selecting and guiding device. The light selecting and guiding device can be positioned to a) direct the excitation light toward the fluid droplet arrangeable on the fluid contact surface of the substrate and b) to direct light inclining from the fluid droplet arrangeable on the fluid contact surface of the substrate to the detection module. The apparatus can comprise at least one or at least two or at least three or at least four optical units. It is also possible that the apparatus comprises more than four optical units.

In one embodiment, the excitation light source can be an excitation filter adapted to filter light of at least one specific wavelength received from a light emitting system. In one embodiment, the excitation filter is either a single band excitation filter or a dualband excitation filter. An excitation filter is a high quality optical-glass filter, which can be used for example in fluorescence microscopy and spectroscopy applications. An excitation filter is used for selection of the at least one excitation wavelength of light from a light source. Some excitation filters select light of relatively short wavelengths from an excitation light source. The excitation filters used may come in 2 main types—short pass filters and band pass filters. Other forms of excitation filters include monochromators, wedge prisms coupled with a narrow slit (for selection of the excitation light) and the use of holographic diffraction gratings, etc.

Thus, the filters used herein allow filtering light from a common light source or from different light sources to select a desired wavelength which is used to excite compositions or optical active molecules which are comprised in a fluid droplet at the fluid contact surface of the substrate.

The apparatus is designed so that the excitation light source or the excitation filter is removably arranged within the rotatable platform of the apparatus. Thus, depending on the application they can be replaced with an excitation light source or an excitation filter providing excitation light of a wavelength(s) suitable for the desired application.

In order to more specifically select the emission wavelength of the light emitted from the composition or the optical active molecule and to remove traces of excitation light before detection, an emission filter can be used. Emission filters are usually a special type of filter referred to also as interference filter, because of the way in which they block the out of band transmission. Interference filters exhibit an extremely low transmission outside of their characteristic bandpass. Thus, they are very efficient in selecting the desired excitation and emission wavelengths.

Therefore, in one embodiment, the detection module comprised in the apparatus described herein can be an emission filter adapted to filter light of a specific wavelength emitted by a composition comprised in a fluid droplet arrangeable on the fluid contact surface of the substrate. In one embodiment a dualband emission filter is used capable of filter light of at least two different wavelengths.

The light selecting and guiding device has the function to direct light from the excitation light source towards the fluid contact surface of the substrate while stopping excitation light from reaching the photo detector. Therefore, in one embodiment a dichroic beam splitter (also called dichroic mirror) is used. In another embodiment, a dualband dichroic beam splitter (also called dualband dichroic mirror) is used. A multilayer of coating of dielectrics enables a dichroic beam splitter to reflect a specific wavelength region and transmit other regions. In the application of the apparatus described herein, the dichroic beam splitter reflects the excitation light towards the fluid contact surface and transmits light emitted by a composition or optical active molecule comprised in a sample located at the fluid contact surface of the substrate. ‘Dichroic’ beam splitters differ from a typical beam splitter in that the light beams are combined or separated without a large loss in the intensity of either light beam, i.e. the excitation light and the light emitted by the composition or optical active molecule comprised in a sample located at the fluid contact surface of the substrate. Also, a beam splitter is usually placed at an angel of incidence for the inclining light of 45°.

A beam splitter can be fabricated for example from a hard-coated ion-beam-sputtered thin coating that is placed on a simple ultra-low-autofluorescence fused-silica substrate without using any adhesives. The dielectric glass coatings are as hard as the glass substrate itself with a scratch-dig of 40-20. Additionally, a beam splitter is virtually impervious to humidity-induced shifting. The uniformity and high flatness of the coated glass avoids unwanted wavefront distortions. The filters can be cleaned and handled like standard glass optics. They withstand high optical irradiation intensities with no noticeable degradation or burn out, even with prolonged use and exposure to ultraviolet light.

In one embodiment of the apparatus described herein, the beam splitter is positioned between excitation light source, substrate and detection module. In this position, the filter functions to both select the emission wavelength(s) of the light emitted by the composition or optical active molecule comprised in a sample located at the fluid contact surface of the substrate and to eliminate any trace of the wavelengths used for excitation via the excitation light source.

Like the excitation light source, the detection module and the light selecting and guiding device can be positioned in the optical unit to be removable. This allows replacing these components for adapting the optical unit to the respective application.

In one embodiment, the apparatus includes one or more optical units using dualband excitation light source, such as a dualband excitation filter, a dualband light selecting and guiding device, such as a dualband dichcroic mirror, and a dualband detection module, such as a dualband emission filter. In another embodiment, the apparatus includes at least two optical units using singleband excitation light source, such as a singleband excitation filter, a singleband light selecting and guiding device, such as a singleband dichcroic mirror, and a singleband detection module, such as a singleband emission filter. Use of dualband systems allows to simultaneously excite two different molecules in a fluid droplet compared to only one molecule when using a singleband system. A singleband system directs light of one specific wavelength through the optical unit while a dualband system directs light of two different wavelengths through the optical unit. To prevent interference of the light of two different wavelengths they can be modulated at different frequencies.

Using a dualband system allows using one singleband optical unit less. In other words, for a dualband system, every single optical unit can be used to excite two optical active molecules in one fluid droplet at the same time while an optical unit directing light of only one specific wavelength can excite only one optical active molecule comprised in a fluid droplet at the surface of the fluid contact surface of the apparatus. Thus, the use of a dualband system allows to further increase the performance of the apparatus of the present invention.

The optical unit(s) can further comprise lenses to assist directing and focusing the light within the optical unit. For example, in one embodiment the optical unit comprises a lens positioned on the side of the detection module which is opposite the side on which the light selecting and guiding device is positioned. The lens is to focus incident light coming from the detection module onto a photo detector. In one embodiment, this lens can also be positioned directly above the photo detector and thus would not form part of the optical unit but part of the support structure also carrying the photo detector and the light emitting system. In that case only one lens directly positioned in front of the photo detector would be needed and none of the optical units would need to carry a lens which is positioned on the side of the detection module which is opposite the side on which the light selecting and guiding device is positioned.

In a further embodiment, another lens in the optical unit can be positioned to focus excitation light received from the light selecting and guiding device onto the fluid contact surface or the fluid droplet arrangeable on the fluid contact surface of the substrate and positioned directly at the position where the excitation light passes through the fluid contact surface and into the fluid droplet.

The lenses can be any lenses known in the art and which can be used to carry out the above functions. In one embodiment, aspheric lenses are used. Aspheric lenses are any lenses whose surface angles and profiles are neither part of a sphere nor part of a cylinder. Conventional or non-aspheric lenses have the ‘same curve across their entire surface, like a ping-pong ball. Aspheric lenses accomplish the same amount of refraction but are flatter and slimmer. The more complex surface profile of an aspheric lens can reduce or eliminate spherical aberration and also reduce other optical aberrations compared to a simple lens. A single aspheric lens can replace a much more complex multi-lens system. The resulting device is smaller and lighter, and often cheaper than a multi-lens design.

The at least one magnetic field generator which is vertically movable parallel to the rotational axis of the rotatable platform can be a magnet, such as a permanent magnet. In one embodiment, the magnetic field of the magnet used in the apparatus of the present invention is adapted to overcome the friction force and surface tension of a fluid droplet. In case the magnetic field generator is to move a magnetizable substance at the fluid contact surface of the substrate, the magnetic field generator is moved into its upper position in which it is closest to the side of the substrate opposite the site of the substrate with the fluid contact surface. In case the magnetic field generator is not supposed to move any magnetizable substance at the fluid contact surface of the substrate it is moved into its lower position.

In one embodiment, the magnetic field generator has a shape adapted to concentrate the magnetic force on a spot at the fluid contact surface of the substrate. Suitable magnet shapes include magnets with a conical shape, wherein the tip of the conical magnet is directed towards the substrate, or a cubical magnet with a hole in its middle. Like for a conical shaped magnetic field generator, the hole will affect the magnetic flux line to concentrate in a spot shortly above the hole. A person skilled in the art will know how to choose the distance between magnetic field generator and substrate to ensure that the strongest point of the magnetic flux line concentrates directly in a sport at the fluid contact surface of the magnet. In one example magnetic force-distance measurements have been carried out with a cone shaped permanent magnet and a magnet comprised of two stapled discs (see FIG. 15). Based on these results, in one embodiment, the magnetic field strength at the fluid contact surface is preferably equal or about 400 mT to ensure that a fluid droplet at the fluid contact surface is movable. For example, in case of a cone shaped permanent magnet, a magnetic field strength of 400 mT correlates with a distance of about 2 to 3 mm from the tip of the cone shaped magnet.

The apparatus described herein can further comprise a support structure. The support structure can support the substrate, the at least two temperature control modules and the rotatable platform with its components. In case the rotatable platform does not carry a photo detector, the support structure can carry at least one or at least two photo detectors. The at least one photo detector can be arranged in a position relative to the rotatable platform which allows to detect light passing through the detection module of the rotatable platform. Since the platform is rotatable and the support structure has in one embodiment a fixed position relative to the rotatable platform, an optical unit housed in the rotatable platform has to be positioned above the photo detector. Rotation of the platform allows positioning the at least one optical unit of the rotatable platform in direct proximity to the photo detector to allow measurement of the light emitted by the compositions or optical active molecules in the sample and passing through the optical unit.

In case the rotatable platform comprises different optical units which are positioned on different circular orbits, the support structure can carry a further optical detector which is, like the optical unit, positioned closer to the rotational axis of the platform mounted on the support structure. This way at least one or two photo detectors ensure that the signals received from the optical units on the outer circular orbit and the inner circular orbit can be measured at the same time if an optical unit is placed directly above the photo detector.

A photo detector is any electronic device, in particular a photodiode that responds to or measures the intensity of light, such as ultraviolet or infrared radiation or visible light.

The photo detector converts optical energy into electrical energy via the photoelectric effect. Any photo detector known in the art can be used. For example, a photo detector can be a photomultiplier tube (PMT) or phototransistors or an avalanche diode (APD). In one embodiment, a BPW21 blue enhanced photodiode from Siemens was used.

The support structure can further comprise a light emitting system arranged to couple light into the at least one of the at least two optical units. In one embodiment, the light emitting system couples light from a single light source, such as a LED into the at least one optical unit. In another embodiment the light emitting system comprises an array of LEDs, wherein each LED emits light of a different wavelength. For example, the array can comprise five different LEDs to couple light from white, blue, green, orange and red LEDs into at least one of the at least two optical units. In still other embodiment, bicolor light source, such as a bicolor LED is used. It is also possible to use combinations of the aforementioned light sources in the light emitting system. The support structure can also carry more than one light emitting system in case it is desired to carry out different measurements at different optical units of the rotatable platform at the same time. In one embodiment one light emitting system is provided which couples light into the optical unit of the rotatable platform. Each respective optical unit is placed in close proximity to the light emitting system by moving the rotatable platform in a position which aligns the position of the excitation light source of the optical unit with the position of the light emitting system. If necessary it is possible to provide a lens positioned between the light emitting system and the excitation light source of an optical unit for focusing and coupling light into the optical unit. The lens can be an aspheric lens. The lens can either form part of the support structure and the light emitting system or can form part of the optical unit and is positioned on the side of the excitation light source opposite the side where the light selecting and guiding device is positioned, i.e. the excitation light source is positioned between lens and light selecting and guiding device.

The support structure can further comprise an actuating device, such as a stepper motor which is connected to the rotatable platform and moves the rotatable platform in clockwise and/or anticlockwise direction around its central axis.

The apparatus also comprises at least one temperature control module. A temperature control module can regulate the temperature of a biological and/or chemical sample located within the temperature zone. The temperature within the temperature zone is controlled by the temperature control module. The size and shape of the temperature zone located at the fluid contact surface of the substrate depends on the size and shape of the temperature control module.

In one embodiment, the apparatus described herein includes at least two temperature control modules. In yet a further embodiment, the apparatus includes a plurality of temperature control modules. In one embodiment the apparatus comprises at least four temperature control modules. Where the apparatus includes more than one temperature control module, they are typically thermally isolated from each other. Such isolation may be achieved by separating the temperature control modules by material that is a poor heat conductor, such as e.g. plastic, wood, glass, quartz, water, air or ceramic.

Where desired, the apparatus may include further means of temperature control, such as a cooling module. Additionally or alternatively, the temperature control module may include a cooler, which is for instance adapted to thermally communicate with the heat conductor. In many embodiments where it is desired to handle a sample at temperature values that are about at or above room temperature, cooling the sample from a higher to a lower temperature value, e.g. from 94° C. to 55° C., can conveniently be achieved without a cooler. The apparatus of the invention can easily be designed to allow for heat emission from the heat conductor and the sample that provides fast cooling rates (e.g. FIG. 11).

The temperature control module—or at least one of the temperature control modules of at least one or two and in some embodiments each of these temperature control modules—is based on a direct heating system in that it includes a heater and a temperature sensor. It furthermore includes a heat conductor. The heater is adapted to thermally communicate with the heat conductor, thus being able to heat the heat conductor. As an illustrative example, the heater may contact the heat conductor. Under the control of the temperature sensor the heater is thereby able to heat the heat conductor up to a desired temperature and/or keep the heat conductor at a desired temperature value. Furthermore, a reduction of the temperature value to which the heater is to heat the heat conductor, usually leads effectively to a decrease in the temperature of the same and may be defined as “cooling”. Typically the temperature sensor is arranged to be able to communicate with the heat conductor, for example via direct contact. The heat conductor may be of any material that is able to conduct heat. The heat conductor may for example include a metal, a semiconductor, a diamond, a carbon nanotube or a fullerene compound. Examples of suitable metals include, but are not limited to, silver, copper, aluminium, zinc, gold, platinum, titanium, iron, lead, nickel, iridium and cadmium. Two illustrative examples of suitable semiconductors are silicon and germanium. Silver and silicon are two typical examples of a heat conductor with a conductivity of 410 Wm⁻¹K⁻¹ and 157 Wm⁻¹K⁻¹, respectively.

The heater, the sensor and the heat conductor may be of any shape and arranged in any orientation with respect to each other. In some embodiments the heater and the heat conductor are arranged in the same plane. In some of these embodiments the heater and the sensor are arranged in direct vicinity to each other.

The apparatus of the invention is designed in such a way that the substrate with the fluid contact surface is situated above the temperature control module. The terms “above” and “below” as used herein, refer to a position, where the apparatus described herein is held in such a way that the substrate may be placed in direct proximity of the temperature control modules and once positioned can be secured solely by the force of gravitation. In some embodiments, the heater is located below the heat conductor. In some embodiments both the heater and the sensor are located below the heat conductor.

In some embodiments the heater includes a surface that is arranged essentially parallel to the fluid contact surface of the substrate, on which fluid contact surface of the substrate the fluid droplet can be placed. In some embodiments the heater includes a surface that is arranged essentially parallel to the fluid contact surface of the substrate, on which fluid contact surface of the substrate the sample is placed. In some embodiments both the heater and the sensor include a surface that is arranged essentially parallel to the fluid contact surface of the substrate, on which fluid contact surface of the substrate the sample is placed. In some of these embodiments the heater and the sensor each comprise a surface arranged in a common plane. This common plane is thus essentially parallel to the fluid contact surface of the substrate, on which fluid contact surface of the substrate the sample is placed. In any of these embodiments the heater, the sensor or both may be located below the heat conductor.

In any of these embodiments, in particular where the heater and the sensor each comprise a surface arranged in a common plane, the heater or the sensor may be concentric. In some embodiments both the heater and the sensor are concentric. One or both of them, or parts thereof, may for instance have the shape of a hollow circle, a hollow rectangle, a hollow triangle, a hollow square, or any hollow or any oligoedron (e.g. FIG. 7 for examples). In one embodiment both the heater and the sensor are concentric and the heater surrounds the sensor. In another embodiment both the heater and the sensor are concentric and the sensor surrounds the heater. In an embodiment, which is depicted in a cross-section in FIG. 9, both the heater and the sensor are concentric and arranged under a concentric heat conductor. It should be noted that in the depicted embodiment the heater, the sensor and the heat conductor include a central hollow area, so that they each appear as respective pairs.

In one embodiment, the temperature control module is arranged or positioned between the substrate and the rotatable platform or to be more precise between the optical unit of the rotatable platform and the substrate. Thus, a temperature control module is generally designed to allow passage of light from and to the optical unit. In one embodiment, the components of the temperature control module, such as heater, heat conductor and temperature sensor are arranged to allow passage of light, such as excitation light or emission light, from and to the fluid contact surface. In another embodiment the temperature control module comprises a concentric hole in the middle which allows passage of light.

In a further embodiment, the temperature control module forms an integral part of the substrate which means that the temperature control module is build into or embedded in the substrate. In such an integrated form the shape and design of the temperature control module can still be the same as in embodiments in which a temperature control module is arranged between optical unit and substrate.

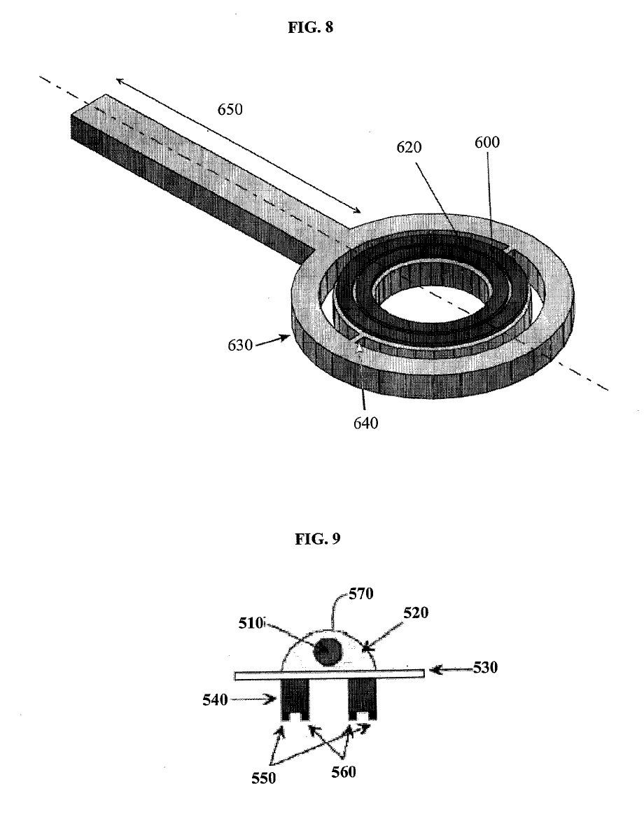

In one embodiment the heat conductor or a part thereof, is of a shape that is adapted to match the shape of the sensor and/or the heater. Where the sensor and the heater are for instance of a square or round concentric shape with a hollow centre, the heat conductor may possess a corresponding square or round concentric shape with a hollow centre. Where a part of the heat conductor is adapted to match the shape of the sensor and/or the heater, it may include additional other parts of any desired shape. As an illustrative example, it may include a rod-shaped part. Where for instance the part of the heat conductor, which is adapted to match the shape of the sensor and/or the heater, is of circular profile, the heat conductor may be of donut shape. FIG. 8 depicts an exemplary embodiment, in which the heat conductor includes two concentric parts, which are connected by a linker. The inner of these concentric parts is in direct contact with a concentric sensor and a concentric heater, the latter surrounding the sensor. The heat conductor furthermore includes a rod-shaped part. It can thus be considered as a double donut shape. Thermal conductance is given by the material of heat conductor, the length of the rod-shaped part, and the cross-section of the concentric parts. Thermal capacitance is given by the double donut volume (FIG. 8) with the volume of the sample.

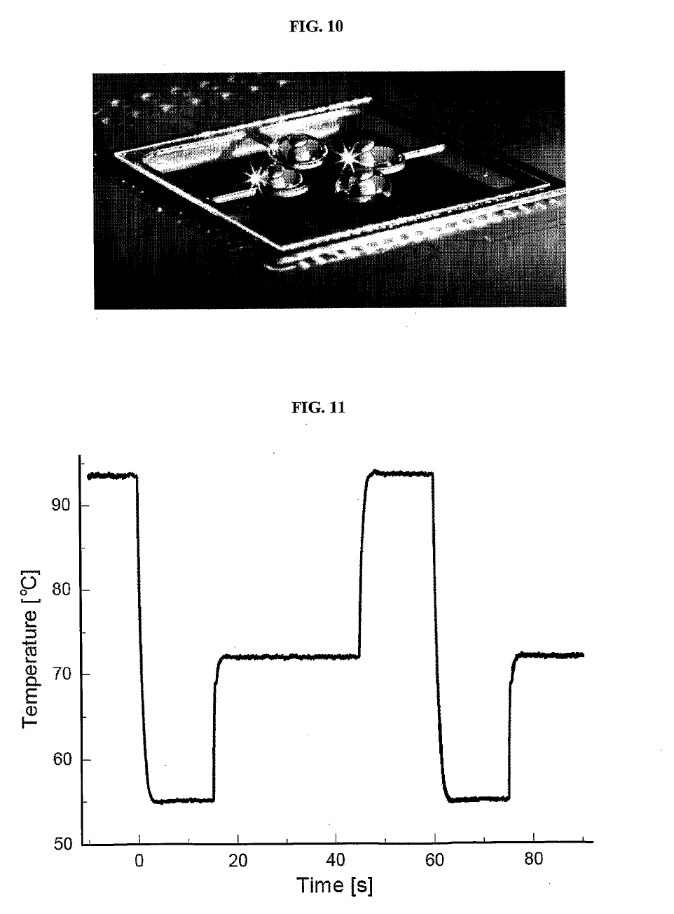

FIG. 10 depicts an arrangement with a glass cover slid as a substrate and fluid droplets placed thereon. The samples shown in FIG. 10 are water based droplets each with a volume of 1 μl and placed directly above the temperature control modules, which are located on the other side of the substrate. The water droplets in the embodiment shown in FIG. 10 are covered with 5 μl of mineral oil.

Before turning to the structure and composition of the substrate and the fluid contact surface, information are provided with respect to the biological and/or chemical sample arrangeable on the fluid contact surface of the substrate. Since the form of application of the biological and/or chemical sample at the fluid contact surface of the substrate determines the required properties of the substrate and the fluid contact surface the types of sample and the way to prepare them are described.

The biological and/or chemical sample may be of any origin. It may for instance be derived from human or non-human animals, plants, bacteria, viruses, spores, fungi, or protozoa, or from organic or inorganic material of synthetic or biological origin. Accordingly, samples including, but not limited to a soil sample, an air sample, an environmental sample, a cell culture sample, a bone marrow sample, a rainwater, a fallout sample, a sewage sample, a ground water sample, an abrasion sample, an archaeological sample, a food sample, a blood sample, a serum sample, a plasma sample, an urine sample, a stool sample, a semen sample, a lymphatic fluid sample, a cerebrospinal fluid sample, a nasopharyngeal wash sample, a sputum sample, a mouth swab sample, a throat swab sample, a nasal swab sample, a bronchoalveolar lavage sample, a bronchial secretion sample, a milk sample, an amniotic fluid sample, a biopsy sample, a cancer sample, a tumour sample, a tissue sample, a cell sample, a cell culture sample, a cell lysate sample, a virus culture sample, a nail sample, a hair sample, a skin sample, a forensic sample, an infection sample, a nosocomial infection sample, a production sample, a drug preparation sample, a biological molecule production sample, a protein preparation sample, a lipid preparation sample, a carbohydrate preparation sample, a space sample, an extraterrestrial sample or any combination thereof may be used.

Where desired, a respective sample may have been preprocessed to any degree. As an illustrative example, a tissue sample may have been digested, homogenised or centrifuged prior to being used with the apparatus of the present invention. The sample may furthermore have been prepared in form of a fluid, such as a solution. Examples include, but are not limited to, a solution or a slurry of a nucleotide, a polynucleotide, a nucleic acid, a peptide, a polypeptide, an amino acid, a protein, a synthetic polymer, a biochemical composition, an organic chemical composition, an inorganic chemical composition, a metal, a lipid, a carbohydrate, a combinatory chemistry product, a drug candidate molecule, a drug molecule, a drug metabolite or of any combinations thereof. Further examples include, but are not limited to, a suspension of a metal, a suspension of metal alloy, and a solution of a metal ion or any combination thereof, as well as a suspension of a cell, a virus, a microorganism, a pathogen, a radioactive compound or of any combinations thereof. It is understood that a sample may furthermore include any combination of the aforementioned examples.

Some samples include, or will be expected to include, target matter or a precursor thereof. The target matter may for instance be a cell or a molecule added to or included in the sample, and it may be desired to expose the target matter to heat. As another example, the target matter may be a compound known or theorized to be obtainable from a precursor compound by means of a chemical process that occurs upon increasing the temperature. In this case the sample may for instance include a solution of such precursor compound.

The target matter or precursor thereof may thus be of any nature. Examples include, but are not limited to a nucleotide, an oligonucleotide, a polynucleotide, a nucleic acid, a peptide, a polypeptide, an amino acid, a protein, a synthetic polymer, a biochemical composition, an organic chemical composition, an inorganic chemical composition, a lipid, a carbohydrate, a combinatory chemistry product, a drug candidate molecule, a drug molecule, a drug metabolite, a cell, a virus, a microorganism or any combinations thereof. In embodiments where the target matter is for example a protein, a polypeptide, a peptide, a nucleic acid, a polynucleotide or an oligonucleotide, it may contain an affinity tag. Examples of affinity tags include, but are not limited to biotin, dinitrophenol or digoxigenin.

Where the target matter is a protein, a polypeptide, or a peptide, further examples of an affinity tag include, but are not limited to oligohistidine, polyhistidine, an immunoglobulin domain, maltose-binding protein, glutathione-S-transferase (GST), calmodulin binding peptide (CBP), FLAG′-peptide. Where the target matter is a nucleic acid, a polynucleotide or an oligonucleotide, an affinity tag may furthermore be an oligonucleotide tag. Such an oligonucleotide tag may for instance be used to hybridize to an immobilized oligonucleotide with a complementary sequence. A respective affinity tag may be located within or attached to any part of the target matter. As an illustrative example, it may be operably fused to the amino terminus or to the carboxy terminus of any of the aforementioned exemplary proteins.

The biological and/or chemical sample can be included in a fluid droplet, such as a liquid droplet. As an illustrative example, it may be included in an inner phase of such a fluid droplet. The inner phase of such a droplet can have a volume in the range of about 1 pl to 1 ml, or a volume in the range of about 0.1 nl to about 500 μl, or a volume in the range of about 100 nl to 100 μl or in a range of 1 nl to about 1 μl. Handling of droplets of a volume above 1 ml in air may in some embodiments require further adaptions of the droplet environment. In this regards, the skilled artisan will be aware that when using a droplet of large volume (such as e.g. 2 ml), the respective droplet may split into smaller droplets when contacting a surface. Where such splitting is undesired when using such a droplet on an apparatus described herein, suitable volumes for a droplet of a selected fluid can easily be determined experimentally.

The fluid droplet can include magnetically attractable matter. Typically only one phase of the fluid droplet contains magnetically attractable matter, i.e. either the outer or the inner phase of the fluid droplet. As an illustrative example, in some embodiments a magnetic fluid such as a ferrofluid may be included in the fluid droplet. A ferrofluid is for example commercially available in form of a colloidal suspension of sub-domain magnetically attractable particles in a liquid carrier from Ferrotec (Nashua, N.H., U.S A.). A respective ferrofluid may for instance be based on a non-polar liquid and form the outer phase of a fluid droplet. In this case the inner phase may for instance be an aqueous solution.

As a further illustrative example, an iron-rich bacterium may be included in a phase of the fluid droplet. Many bacterial species contain iron as it is required for their metabolism. A large number, including Neisseria meningitidis and N. gonorrhoeae, have for example transferrin and/or lactoferrin iron-uptake systems. Such bacteria may only in certain embodiments contain sufficient iron to be used as magnetically ‘attratable’ matter and thus being actuatable through the magnetic field generator of the apparatus described herein.

Magnetically attractable particles can be able to attract target matter. In some embodiments the magnetic particles can be functionalized with specific affinity for target matter and capturing target matter, such as specific nucleic acids, therefore acting as a binding means.

Magnetically attractable particles are herein referred to as “magnetic particles” or “magnetic beads”. Magnetic particles may contain diamagnetic, ferromagnetic, paramagnetic or superparamagnetic material. Superparamagnetic material responds to a magnetic field with an induced magnetic field without a resulting permanent magnetization. Magnetic particles based on iron oxide are for example commercially available as Dynabeads® from Dynal Biotech.

The magnetic beads may be designed to serve the function of attracting target matter through chemisorption, e.g. a covalent bond, or physisorption, e.g. electrostatic attraction. The magnetic particles used in such embodiments may provide a surface with an affinity for certain matter allowing for instance to absorb/desorb proteins, peptides, nucleic acids and other compounds. Examples include, but are not limited to, attractions by physical means, such as e.g. π-stacking, dipole-dipole, induced dipole-dipole, van-der-Waals, opposite charges, or H-bonding, e.g. antibody-antigen binding attractions, and affinity attractions formed between a ligand that has binding activity for the target matter and the target, such as for instance a ligand and a metal. As two further illustrative examples, physicochemical bonds, e.g. between gold and a thiol, or geometrical means, e.g. size exclusion, may be relied on. Different areas of the same or several magnetic particles may also be designed to attract or “capture” the target matter.

In some embodiments the magnetic particles include a ligand that is capable of binding target matter that is suspected or known to be included in the biological and/or chemical sample. Such a ligand may in some embodiments be capable of selectively binding such target matter such as, an ion, a polyion, a metal, DNA, RNA, a protein (including a synthetic analogue thereof), bacterial cells, spores, viruses, low molecular weight organic molecules, or inorganic compounds. A respective ligand may be immobilized on the surface of the at least one magnetically attractable particle.

A respective ligand may for instance be hydrocarbon-based (including polymeric) and include nitrogen-, phosphorus-, sulphur-, carben-, halogen- or pseudohalogen groups. It may be an alcohol, an organic acid, an inorganic acid, an amine, a phosphine, a thiol, a disulfide, an alkane, an amino acid, a peptide, an oligopeptide, a polypeptide, a protein, a nucleic acid, a lipid, a saccharide, an oligosaccharide, or a polysaccharide. As further examples, it may also be a cation, an anion, a polycation, a polyanion, a polycation, an electrolyte, a polyelectrolyte, a carbon nanotube, carbon nanofoam, a silica particle, a glass particle, or an alumosilicate. Generally, such a ligand has a higher affinity to the target matter than to other matter.

Examples of a respective ligand include, but are not limited to, a crown ether, an antibody, a fragment thereof and a proteinaceous binding molecule with antibodylike functions. Examples of (recombinant) antibody fragments are Fab fragments, Fv fragments, single-chain Fv fragments (scFv), diabodies or domain antibodies. An example of a proteinaceous binding molecule with antibody-like functions is a mutein based on a polypeptide of the lipocalin family. Further examples of a suitable ligand include, but are not limited to, a molecular imprinted structure, an extracellular matrix, a lectin, protein A, protein G, a metal, a metal ion, nitrilo triacetic acid derivates (NTA), RGD-motifs, dextranes, polyethyleneimine (PEI), polyelectrolytes, redoxpolymers, glycoproteins, aptamers, enzymes, a dye, streptavidin, amylose, maltose, cellulose, chitin, glutathione, calmodulin, gelatine, polymyxin, heparin, NAD, NADP, lysine, arginine, benzamidine, poly U, or oligo-dT. Lectins such as Concavalin A are known to bind to polysaccharides and glycosylated proteins. An illustrative example of a dye is a triazene dye such as Cibacron blue F3G-A (C13) or Red HE-3B, which specifically bind NADHdependent enzymes.

In some embodiments the target matter is a molecule that is suspected or known to be present within other (undesired) matter, from which it needs to be extracted. Extraction of a molecule from an organism, a part of an organism, or an embryo may for instance include the usage of a compound that facilitates the transfer of a desired molecule from an organism or a part thereof into a fluid. An illustrative example of an extraction of a molecule from a part of an organism is an extraction of proteins (wholly or partly) integrated into the cell membrane. It is often desired to transfer such proteins into an aqueous solution for further processing. A compound that facilitates the transfer of such proteins into an aqueous solution is a detergent. Contacting a respective cell membrane with an aqueous solution, to which a detergent is added, will typically result in an extraction of membrane proteins. Where magnetic particles are used, they may at the same time as acting as a carrier for target matter, or alternatively thereto, themselves act as a tag or amplifier in the context of sensor technologies.

As an illustrative example, target matter may be bound to ligands immobilized on different magnetic particles in a fluid droplet. By means of further affinity ligands, whether bound on a stationary phase, in solution, or otherwise the target matter may be separated together with the magnetic particles bound thereto. Where the magnetic particles are exposed to a magnetic field, they develop a dipole field. This dipole field may be detected by a dipole sensor. By quantifying the amplitude of the sensor impedance the amount of target matter can be quantified. The fluid droplet further includes an inner phase and an outer phase. The outer phase is surrounding the inner phase. In some embodiments the outer phase is a bulk phase accommodating the inner phase. In other embodiments the outer phase is surrounding the inner phase as a film. The fluid of the outer phase may be a liquid or a gas. The fluid of the inner phase is typically a liquid.

In embodiments the outer phase of the fluid droplet is a film. The film is typically of a volume that is in the range of several magnitudes below to several magnitudes above the volume of the inner phase. The volume ratio of the inner to the outer phase may for example be selected in the range of about 1000:1 to about 1:1000, such as the range of about 10:1 to about 1:10. As an example, for applications involving one or more liquid droplets at room temperature it may be desired to chose a high volume ratio of the inner to the outer phase, for instance a ratio of about 1000:1. For applications involving one or more liquid droplets in the range of about 100° C. it may be desired to choose a low volume ratio of the inner to the outer phase, for instance a ratio of about 1:1000.

The outer phase is immiscible with the inner phase. Typically, the fluid of the outer phase is immiscible with the fluid of the inner phase. Any fluid may be used for the respective phase, as long as it is (a) immiscible with the other phase, so that two separate phases can form, and (b) the fluid does not prevent the desired process from being carried out.

Chemical and biochemical processes are typically carried out in the inner phase, respectively. Thus a selected fluid may be of any property. In case a phase is selected to be a liquid or a gas, it may for instance be a polar or a non-polar liquid or gas, respectively. Often liquids are classified into polar and non-polar liquids in order to characterize properties such as solubility and miscibility with other liquids.

Examples of non-polar liquids include, but are not limited to hexane, heptane, cyclohexane, benzene, toluene, dichloromethane, carbon tetrachloride, carbon disulfide, dioxane, diethyl ether, or diisopropylether. Examples of dipolar aprotic liquids which can also be used are methyl ethyl ketone, chloroform, tetrahydrofuran, ethylene glycol monobutyl ether, pyridine, or dimethylsulfoxide, to name only a few. Examples of polar protic liquids are water, methanol, isopropanol, tert.-butyl alcohol, formic acid, hydrochloric acid, sulfuric acid, acetic acid, trifluoroacetic acid or chlorophenol.

Two immiscible phases may for instance be obtained where a polar fluid, such as a hydrophilic liquid, is selected for one phase and non-polar fluid, such as a hydrophobic liquid, is selected for the other phase. In some embodiments the fluid of the inner phase may be a polar liquid and the fluid of the outer phase of the fluid droplet may be a nonpolar liquid. Suitable polar liquids include, but are not limited to, water, deuterium oxide, tritium oxide, an alcohol, an organic acid (including a salt thereof), an inorganic acid (including a salt thereof), an ester of an organic acid, an ester of an inorganic acid, an ether, an amine (including a salt thereof), an amide, a nitrile, a ketone, an ionic detergent, a nonionic detergent, carbon dioxide, dimethyl sulfone, dimethyl sulfoxide, a thiol, a disulfide, or a polar ionic liquid.

As an illustrative example, the fluid of the inner phase of the fluid droplet may be hydrophilic liquid and the fluid of the outer phase of the fluid droplet may be a hydrophobic liquid. Hydrophilic (“water-loving”) liquids, also termed lipophilic (“fat loving”), contain molecules which can form dipole-dipole interactions with water molecules and thus dissolve therein. Hydrophilic (“water-hating”) liquids, also termed lipophobic, have a tendency to separate from water. Examples of a hydrophilic liquid include, but are not limited to water, acetone, methanol, ethanol, propanol, isopropanol, butanol, tetrahydrofuran, pyridine, chloroform, ethylene glycol monobutyl ether, or pyridine, to name only a few.

Examples of a polar ionic liquid include, but are not limited to 1-ethyl-3-methylimidazolium tetrafluoroborate, N-butyl-4-methylpyridinium tetrafluoroborate, 1,3-dialkylimidazolium-tetrafluoroborate, or 1,3-dialkylimidazolium-hexaffuoroborate, to name only a few. Examples of a non-polar liquid include, but are not limited to mineral oil, hexane, heptane, cyclohexane, or benzene, to name only a few.

Examples of a non-polar ionic liquid include, but are not limited to, 1-ethyl-3-methylimidazolium bis[(trifluoromethyl)-sulfonyl]amide bis(triflyl)amide, or 1-ethyl-3-methylimidazolium bis[(trifluoromethyl)-sulfonyl]amide trifluoroacetate, to name only a few.

A phase of the fluid droplet may include further matter, for example dissolved, emulsified or suspended therein. As an illustrative example, where an aqueous phase is used, it may include one or more buffer compounds. Numerous buffer compounds are used in the art and may be used to carry out the various processes.

Further examples of matter included in a phase of the fluid droplet include, but are not limited to, reagents, catalysts and reactants, for carrying out a chemical or biological process. As an illustrative example, salts, substrates or detergents may be added in order to maintain cells or proteins in an intact state. As a further illustrative example, chelating compounds may be required, for instance to protect organisms from traces of otherwise toxic salts or to increase the yield of a chemical reaction. As yet further illustrative examples, protease, RNase, or DNase inhibitors may be added in order to maintain proteins, RNA, or DNA in an intact state. A further example of a possible additive to a phase of the fluid droplet includes magnetically attractable particles (supra).

The inner phase of the fluid droplet can be shielded from the environment by the outer phase. The outer phase may thus for example act as a barrier or as a seal. The term “environment” refers to any fluid or solid matter, such as for instance a gas (of any desired density or pressure) or a liquid, which is not part of the inner phase, the outer phase or a surface, on which the fluid droplet is disposed, such as the fluid contact surface of the substrate.

As an illustrative example, the outer phase may prevent or reduce evaporation of the inner phase into surrounding air. As a further example, the outer phase may provide a barrier in terms of contact or diffusion etc. The outer phase may for instance prevent contact with solid matter such as sand or dust particles or with fluid that would be miscible with the inner phase of the fluid droplet.

The outer phase may also serve in protecting a surface at which the fluid droplet is positioned against contamination by components of the inner phase of the fluid droplet. Furthermore, the outer phase may enable a sample such as a body liquid, e.g. blood, sputum, etc. to move on a non-polar fluid contact surface (e.g. PTFE). In some embodiments the outer phase may also maintain sterility of the inner phase, even where the fluid droplet as a whole is being handled under, or exposed to, non-sterile conditions. The outer phase may furthermore allow for the contact and fusion with another fluid droplet that includes two phases of similar polarities (e.g. similar hydrophobicities). As an example, where the outer phase is a hydrophobic liquid and the inner phase is a hydrophilic liquid, the outer phase may be capable of merging with the outer phase of a further fluid droplet that is hydrophilic and surrounds an inner phase that is hydrophilic. In such a case a spontaneous fusion of the two exemplary droplets may occur.

As mentioned above, the inner phase of the droplet may directly contact matter that is included in at least one fluid contact surface on which the droplet is or is intended to be disposed. Two illustrative examples of such matter are a solid surface or the surface of a fluid. The at least one fluid contact surface of the substrate may have any shape and geometry as long as it is of such a texture, e.g. roughness and waviness, that the fluid droplet remains intact upon being contacted therewith.

As an illustrative example, it will typically be required to provide a surface with a roughness for the fluid of the inner phase of the fluid droplet that is low enough to allow a fluid droplet that gets in contact therewith to remain intact. The term “intact” refers to the existence of a defined droplet including two phases. The fluid droplet is thus understood to remain intact, while it is for instance spread to a desired extend, or merged with another droplet.

Where for instance the inner phase of the fluid droplet is a polar liquid, such as an aqueous fluid, the at least one fluid contact surface of the substrate may be non-polar. In one embodiment the inner phase of the fluid droplet is an aqueous fluid, e.g. water, and the at least one surface is non polar. A respective non-polar surface can include, but is not limited to silicone (including surface-modified silicone), a polymer such as plastic (whether a biopolymer or a synthetic polymer, including a partially fluorinated polymer, a perfluorinated polymer, and a surface-modified polymer), surface-modified silicon oxide, surface-modified silicon hydride, surface-modified paper, surface-modified glass such as e.g. surface-modified pyrex, surface-modified quartz, surface-modified glimmer, surface-modified metal, surface-modified alloy, surface-modified metal oxide, surface-modified ceramic, and any composite thereof. As a further illustrative example, the inner phase of the fluid droplet may be hydrophilic and the at least one surface may be hydrophobic or oleophobic. As yet another illustrative example, the inner phase of the fluid droplet may be non-polar and the at least one surface may be polar.

A surface modification is typically obtained by a treatment carried out to alter characteristics of a solid surface. Such a treatment may include various means as they are described in detail for example in WO 2007/094739 A1.

Where a method according to the present invention is to be combined with another method such as an analytical or preparative method (see also below), it may be desired to provide a surface that allows, or is advantageous for, carrying out both such a further method and a method according to the present invention. During, or before, carrying out such a further method the integrity of the two phases of the fluid droplet may be affected or degraded. As a consequence matter that is located in the inner phase of the fluid droplet may be exposed to another fluid phase and contact the surface. The availability of various suitable inner and outer phases for the fluid droplet used in the present invention typically allows for a flexible selection of a chemical surface treatment, including a coating.

For example, it may be desired to perform an electrophoretic separation or an isoelectric focusing, for instance by subjecting the magnetic particles, whether included in the fluid droplet or not, thereto. It may for instance be desired to provide a surface with minimal interactions for any matter present, which is detectable by the selected method. Where it is for instance desired to analyse the purity of an isolated protein by applying an electromagnetic field (such as an electrophoretic method), analysis results may be falsified by a surface that significantly interacts with proteins. Two illustrative example of a suitable surface coating with minimal protein interactions are the polar polymer poly-N-hydroxyethylacrylaxnide and poly(ethylene glycol)-terminated alkyltrichlorosilane. It is likewise known that the properties of a surface of a device used for isoelectric focusing affect the efficiency for obtaining narrow isolated zones during both the focusing and mobilization processes.

Furthermore, the at least one surface may provide areas of different surface characteristics. In the above illustrative example of an inner phase of the fluid droplet being a polar (e.g. hydrophilic) liquid, some areas of the surface may for example be more non-polar (e.g. hydrophobic) than others, or some regions may be polar (e.g. hydrophilic). As an illustrative example, a surface area of increased polarity may be desired to achieve a spreading of a droplet on a DNA-array for hybridization. Any part of the at least one surface may also be treated in such a way that it provides respective polar or non-polar surface characteristics. For example a solid surface may be treated respectively. A common way of defining the wettability of a surface for a fluid such as a liquid is the contact angle (also termed wetting angle) between a droplet of the fluid in thermal equilibrium on a horizontal surface, which is generally smooth and homogeneous, typically surrounded by a gas such as air. In this respect, a person skilled in the art will be aware of the fact that an increasing roughness of a surface typically increases the contact angle.

In some embodiments the fluid contact surface of the substrate, e.g. a solid surface, is furthermore inert against the fluid of the inner or the outer phase of the fluid droplet. Such embodiments allow for multiple reusing of the device. An illustrative example of a material that is inert against most corrosive media is a fluoropolymer such as fluoroethylenepropylene (FEP), polytetrafluoroethylene (PFTE, Teflon), ethylene-tetrafluoroethylene (ETFE), tetrafluoroethylene-perfluoro-methylvinylether (MFA), vinylidene fluoride-hexafluoropropylene copolymer, tetrafluoroethylene-hexafluoropropylene copolymer, vinylidene fluoride-hexafluoropropylene-tetrafluoroethylene terpolymer, perfluoromethyl vinyl ethertetrafluoroethylen copolymer, perfluoroalkoxy copolymer (PFA), poly(vinyl fluoride), polychlorotrifluoroethylene, fluorosilicones, or fluorophosphazenes. Further examples include, but are not limited to silanes and optically transparent (thermally-conductive) bulk polymer materials with a fairly high heat deflection temperature. An example for a silane includes, but is not limited to mono- or multilayers on quartz, glass or LiNbO₃-surfaces. Examples of optically transparent bulk polymer materials include, but are not limited to polypropylene, a cyclic olefin, liquid crystal polymer, polyvinyl chloride, and the like, and combinations thereof. In form of a thin film, such as a plasma deposited thin film) all of these materials are light transmissive.

The fluid contact surface and the substrate can be made of the same or different of the aforementioned materials. In one embodiment the material used for the fluid contact surface and the substrate is made of a light transmissive material which allows excitation light and emission light to freely pass through it.

Controlling the position of the fluid droplet relative to the at least one fluid contact surface of the substrate further includes exposing the fluid droplet to a magnetic or an electromagnetic field via the magnetic field generator. This exerts a force on the magnetic particles, such that the droplet as a whole is forced to follow any movement of the magnetic particles. Thereby the position of the fluid droplet can be controlled.

In some embodiments a constant magnetic or electromagnetic field is applied, while in other embodiments the magnetic or electromagnetic field is altered during the process. In some embodiments controlling the position of the magnetic field generator allows to move the fluid droplet at the fluid contact surface of the substrate. Thus, rotating the platform including the magnetic field generator allows moving the fluid droplet at the fluid contact surface of the substrate. As a consequence the position of the fluid droplet relative to the fluid contact surface of the substrate can be altered.

In some embodiments several means of controlling the position of a fluid droplet may be combined (cf also below for further examples). In some embodiments the process is only performed once the fluid droplet has been positioned by means of the magnetic or electromagnetic field. In one of these embodiments, the magnetic or electromagnetic field is terminated after the fluid droplet has been placed in a desired position by lowering the magnetic field generator in the rotatable platform.

The apparatus described herein can be used to carry out methods for performing a process on the biological and/or chemical sample in the fluid droplet. Thus, in one aspect the present invention is directed to a method of processing at least one biological and/or chemical sample. The method comprises or consists of disposing at least one fluid droplet onto a fluid contact surface of a substrate of the apparatus described herein. The method further comprises performing a process on the biological and/or chemical sample in the at least one fluid droplet; wherein the fluid droplet comprises an inner phase and an outer phase, and wherein the outer phase is immiscible with the inner phase, and the outer phase is surrounding the inner phase, and wherein the inner phase comprises the biological and/or chemical sample, and the inner phase is shielded from the environment by the outer phase; wherein the fluid droplet comprises magnetically attractable material.

Any process may be performed that can be performed in a fluid droplet. Examples of processes that may be performed include, but are not limited to a physical detection of target matter suspected or known to be included in the sample, a chemical reaction, a cell lysis, an extraction of a molecule from an organism or a part of an organism, a release of a molecule from an organism, and any combination thereof.

Examples of a physical detection include, but are not limited to a spectroscopic, a photochemical, a photometric, a fluorometric, a radiological, an acoustical, an electrochemical, a colourimetrical, a diffractional, an interferometrical, an elipsometrical, and a thermodynamic detection and include for instance the use of photoactive, fluorescent, radioactive or enzymatic labels.

Two illustrative examples of a spectroscopic method are Raman microscopy and coherent anti-Stokes Raman scattering (CARS) microscopy. The latter technique is for example suitable for selective imaging of specific molecules of interest. Examples of a chemical reaction include, but are not limited to a chemical synthesis, a chemical degradation, an enzymatic synthesis, an enzymatic degradation, a chemical modification, an enzymatic modification, an interaction with a binding molecule, and any combination thereof.

Examples of an enzymatic synthesis include, but are not limited to a protein synthesis, a nucleic acid synthesis, a peptide synthesis, a synthesis of a pharmaceutical compound, and any combination thereof.

In embodiments where it is desired to remove matter, such as by-products or undesired matter of the sample, the process may be a washing process or a process including a washing step. It may also include splitting the fluid droplet into at least two daughter fluid droplets. As an illustrative example, a nucleic acid may be extracted from a cell and be bound by a ligand attached to magnetic particles, while cell debris and reagents are to be discarded.

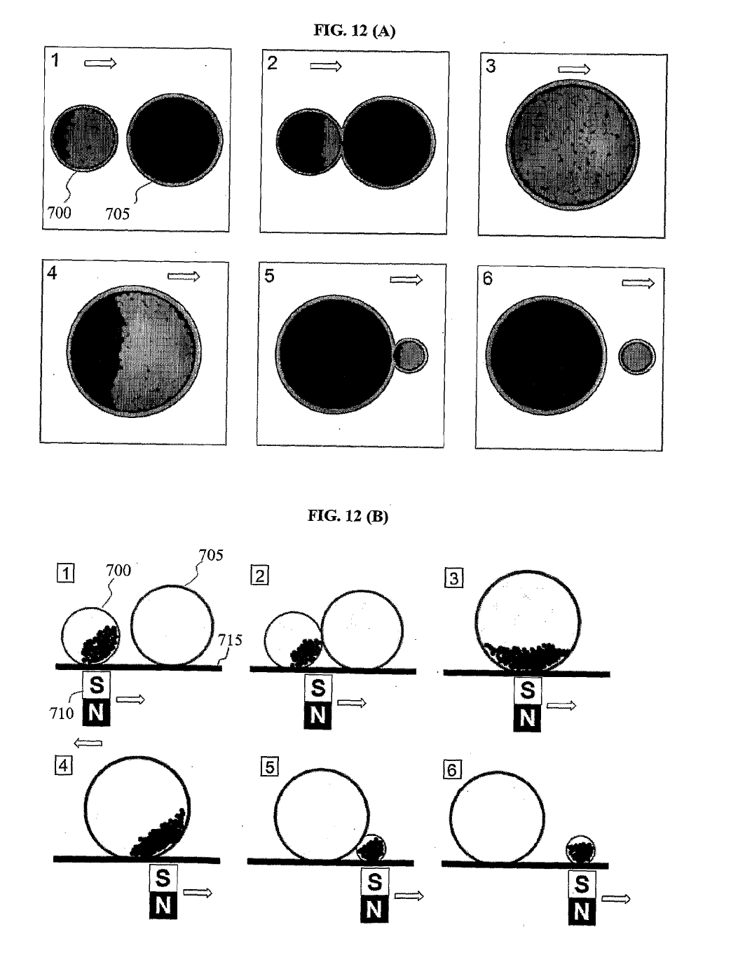

FIG. 12(A) illustrates an example of a washing step of a fluid droplet using a further, additional fluid droplet (FIG. 12(B)). This further fluid droplet may also include two or more fluid phases. It is moved toward the fluid droplet that includes two phases, magnetic matter and the sample (FIG. 12(A) 1). The arrow in FIG. 12A indicates the current position of a permanent magnet. The two fluid droplets merge (FIG. 12(A) 2) and form one larger fluid droplet (FIG. 12(A) 3). To ensure a complete mixing and washing a weak magnetic force may be applied that is sufficient to for instance lift the magnetic particles within the droplet without raising the entire fluid droplet. By further moving the magnetic particles to one side (FIG. 12(A) 4) a splitting of the droplet is initiated (FIG. 12(A) 5). The ratio of magnetic particles/outer phase, the volume ratio of interacting fluid droplets, their biochemical composition, the surface morphology, the surface chemistry, and the strength of the (electro)magnetic field gradient dictate whether the corresponding fluid droplets move, merge, are ‘washed’ or split. During these manipulations the dead volume is zero, i.e. no material is lost even if nanoliter volumes are processed. Where desired, further functional units may easily be implemented in the apparatus of the invention, e.g. piezoelectric based actuators to assist or achieve mixing.

The inner phase of the fluid droplet may be washed or exchanged with any fluid, for instance a solvent, an acid or a base, as long as the fluid allows for (a) the inner phase to remain essentially intact and (b) the magnetic particles to remain attractable to a magnet. In embodiments where the outer phase forms a film surrounding the inner phase (supra), it may furthermore be desired to keep the outer phase intact as a film. In embodiments where a ligand attached to magnetic particles is used to bind target matter, it may furthermore be desired that such a fluid allows for the ligand to remain intact and to bind the desired target matter. At any point in time before, during or after performing such a process, a mixing of the fluid droplet may be carried out, for instance by exposing the fluid droplet to ultrasound. Since the droplet is based on a self-organizing system, such mixing does not affect the integrity of the droplet, but rather assists in achieving an equal distribution of matter within a phase within the droplet. The possibility to perform transfers of matter such as washing allow for complex processes to be performed. Since desired target matter may be bound to ligands immobilized on magnetic particles, the possibility to add, remove or exchange fluid, e.g. liquid, enables the isolation of any matter, e.g. peptides, proteins, DNA, RNA, small organic molecules, metal ions, etc. at any desired stage or step, and complex biochemical transformations can be carried out in sequence 5 (FIG. 12). Furthermore the volume of the fluid droplet can be changed by several orders of magnitude. Accordingly the method described which can be carried out with the apparatus of the present invention provide an interface between the macroscopic and microscopic world without any break in technology.

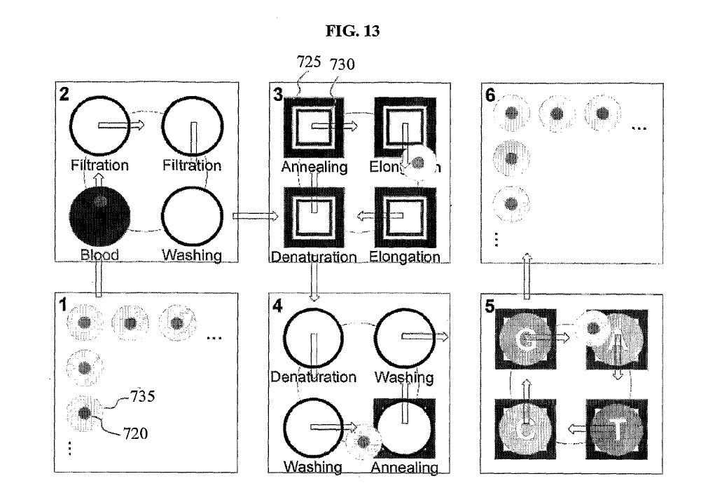

FIG. 13 illustrates a method which can be carried out with the apparatus of the present invention for a polymerase chain reaction. The results of a PCR carried out with the apparatus of the present invention are described in the experimental section. Temperature control can be achieved by means of thin film temperature control modules and temperature sensors which form part of the temperature control module. Module 1 represents a matrix of superparamagnetic particles, which are modified with ligands. These ligands are receptors directed against different cell surface markers. Any cell of interest may in this way be isolated from body fluids or tissue as described above. A drop of capillary whole human blood may be obtained by finger pricking with a lancet. This drop of blood is placed onto Module 2. Leucocytes may then be isolated according to the binding of their cell surface markers to the ligand immobilized on the magnetic particles. Leucocytes can be thermally lysed in the heating zone which is temperature controlled by the thin film temperature control modules of Module 3 (in this case the temperature). The polymerase chain reaction (PCR) is performed in a clock-wise manner by guiding the sample over three different temperature zones or by changing the temperature within one temperature zone to three different temperatures (not shown). FIG. 11 depicts a temperature profile measured using at a temperature zone at the fluid contact surface of the substrate of the apparatus of the present invention. PCR products thus obtained are of a quality that does not differ from a product obtained by conventional methods used in the art. A biotinylated PCR product may be generated, which can be bound to streptavidin coated superparamagnetic particles. The amplification product can be chemically denaturated on Module 4 and annealed to a sequencing primer for pyrosequencing (PSQ), which may be carried out in a clock-wise manner on Module 5 by moving the sample through four different areas at the fluid contact surface containing the bases G, A, T and C. Using time-space conversion makes multiplexing of samples possible. If desired, the samples can be stored in an area of the substrate after pyrosequencing.

Alternatively they can also be processed further. Yet another example of performing a process on the biological and/or chemical sample in a fluid droplet is filtering the fluid droplet through another fluid droplet, as illustrated in FIG. 12. Such a filtration is typically performed by means of moving a smaller droplet containing functionalized superparamagnetic particles with immobilized target matter through a bigger fluid droplet. In this way undesired components such as for example byproducts, impurities, substrates, reagents, solvents or solvent components, salts, enzymes, waste, or buffers, can be diluted in the bigger droplet. Upon further movement of the magnetic particles out of the bigger droplet, essentially only the superparamagnetic particles including the immobilized target matter are being removed from the bigger droplet, while most of the undesired matter is being left behind. Due to the self-organizing nature of the system, the outer phase or a part thereof is likewise removed from, the larger droplet. In case of an outer phase in form of a film, a thin film of the outer phase may for example surround a small remaining amount of inner phase. In this way it is possible to substantially remove matter from the fluid droplet that is not immobilized by the magnetic beads.

The underlying purification effect resembles the mechanism known from affinity chromatography, where target matter is held back by functionalized column material forming the stationary phase, and rinsed/washed several times with a washing solution, forming the mobile phase. In contrast to affinity chromatography, in the method described herein the washing solution is the stationary phase, while the functionalized material is the mobile phase. It should furthermore be noted that no dead volume occurs using such methods.

Furthermore, in contrast to affinity chromatography, the method described herein allows for the elution of target matter in nanoliter volumes. This advantage is crucial in applications such as biosensing, when for example a high concentration of target matter is present in tiny volumes, or where fast kinetics are to be analyzed.

Thus, the apparatus of the present invention can be used for any method described herein. In one aspect the apparatus can be used for carrying out a nucleic acid amplification process. Examples for such nucleic acid amplification processes include, but are not limited to reverse-transcriptase (RT), a polymerase chain reaction (PCR), a real-time quantitative RT-PCR (qRT-PCR), a helicase dependent amplification (tHDA), a smart amplification process (SMAP), a loop-mediated amplification (LAMP), a rolling circle amplification (RCA), or a recombinase polymerase amplification (RPA).

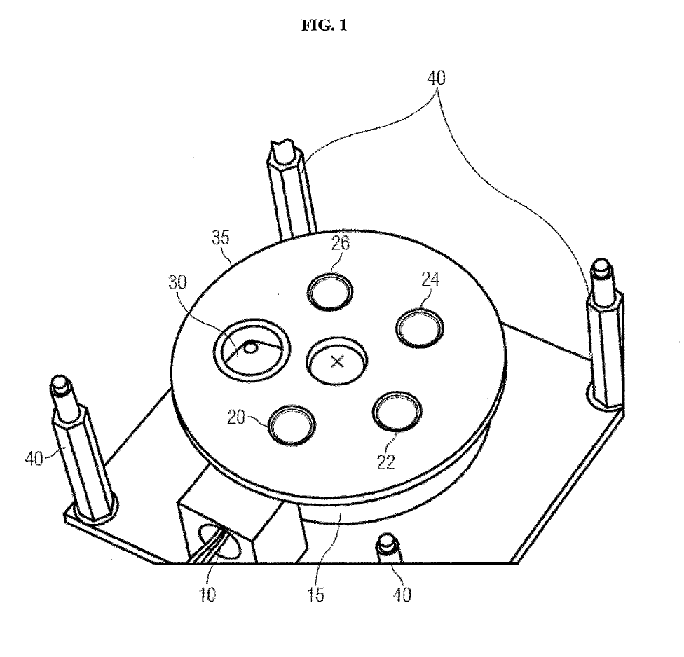

FIGS. 1 to 3 illustrate exemplary embodiments of an apparatus of the present invention.

FIG. 1 shows an oblique view of an apparatus of the present invention without the substrate. In FIG. 1 the rotatable platform 15 is mounted on the support structure 55 via a support shaft located in the center of the rotatable platform 15 (marked with “x”). The support structure 55 also provides some support rods 40 adapted to carry the substrate (not shown). Thus, in general, the support structure can further comprise a holding structure, such as support rods, for mounting the substrate on the apparatus. The substrate is to be positioned directly above the top surface 35 of the rotatable platform.

The embodiment illustrated in FIG. 1 further shows a light emitting system 10 which is located at the side of the rotatable platform 15 and a pair of glass fiber cables directing the light to the light emitting system. The light emitting system 10 couples the light into one of the optical units. The optical unit or units as such cannot be seen in FIG. 1, only the openings within the top surface 35 of the rotatable platform 15 through which light from the optical units is directed to the substrate arrangeable above the platform can be seen 20, 22, 24, 26. FIG. 1 further shows the opening in the top surface of the platform through which the magnetic field generator can be seen. In this embodiment, the magnetic field generator is a magnet with a conical shape 30. The magnet 30 is in its lower position within the platform, i.e. the position in which it would not be able to move any magnetically attractable matter at the fluid contact surface of the substrate arrangeable above the platform.

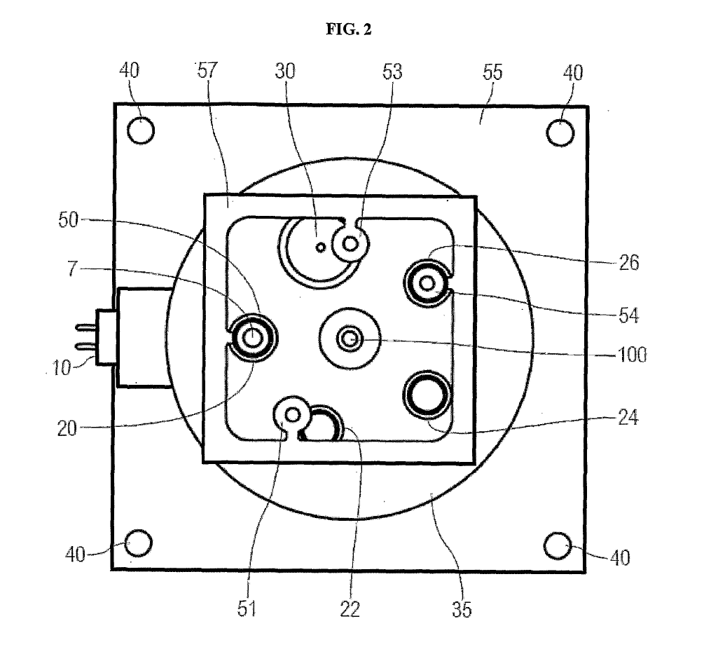

FIG. 2 shows a top view of an embodiment of an apparatus including the support structure 55, the rotatable platform 15 and the substrate. FIG. 2 shows only the fluid contact surface 57 of the substrate. As in FIG. 1, the apparatus illustrated in FIG. 2 has one light emitting system 10 located at the side of the rotatable platform 15. In the position of the rotatable platform 15 shown in FIG. 2, the light emitting system 10 couples light into the optical unit (not shown). The opening 20 in the top surface of the rotatable platform 35 indicates at which point the light from the optical unit is directed towards the substrate. To couple light into one of the optical units which are located below the openings 22, 24 and 26 the rotatable platform needs to be moved into a position which aligns any of the optical units in front of the light emitting system. The rotatable platform can rotate in clockwise as well as anticlockwise direction.

The apparatus shown in FIG. 2 comprises four temperature control modules. Only the temperature control modules 53 and 51 are directly visible while the other two temperature control modules are covered by fluid droplets 54 and 7, 50 which are positioned directly above the temperature control module. The temperature control modules control the temperature in the temperature zone in the fluid contact surface of the substrate, wherein the area of the temperature zone(s) is in this embodiment determined by the shape of the temperature control modules which are arranged directly below the substrate or which form an integral part of the substrate. In this embodiment the temperature control modules are covered with a teflonized glass surface.

The fluid droplet covering the temperature control module which is located directly above the optical unit which is positioned before the light emitting system 10 comprises an outer phase 50 and an inner phase 7. The other droplet 54 is situated directly above the opening 26 to the underlying optical unit. In one embodiment, a fluid droplet is positioned on the fluid contact surface above the temperature control modules 53, 54 or 51. Also shown is a magnetic field generator, which is a conical shaped magnet 30 in the present embodiment. In the support structure 55 the top of the support rods 40 can be seen as well as the central axis 100 with which the rotatable platform 15 is mounted on the support structure 55.

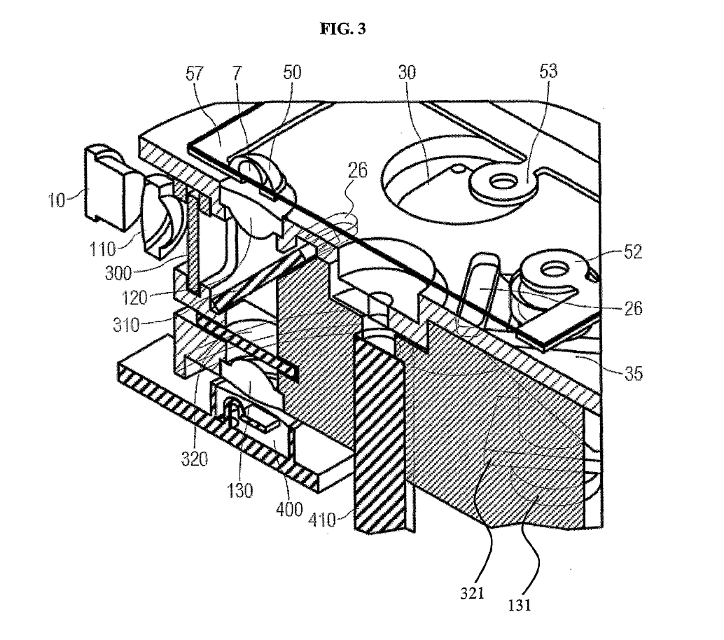

FIG. 3 shows a sectional view of an exemplary embodiment of an apparatus of the present invention. The support structure as well as the rotatable platform and the substrate are shown. Also shown are the light emitting system 10 and the photo detector 400 which are aligned to the optical unit shown in the left half of FIG. 3 to couple light into the optical unit and to detect light coming from the optical unit, respectively.

The optical unit in this embodiment comprises an excitation filter 120 which is positioned before a lens 110 of the light emitting system 10 so that light from the light emitting system 10 can pass through the excitation filter 120. The optical unit further comprises a beam splitter 310 which is positioned in a 45° angle relative to the fluid contact surface 57 of the substrate and the excitation filter to direct light from the excitation filter towards the droplet at the fluid contact surface 57 of the substrate. The fluid droplet comprising an inner phase 7 and an outer phase 50. FIG. 3 also shows an emission filter 320 which is positioned directly next to the lens 130 of the photo detector 400. As can be further seen in FIG. 3 the components of the optical unit, such as excitation filter, beam splitter and emission filter are mounted in a removable manner (slits) in the rotatable platform. For example, the beam splitter 310 can be removed through an opening 26 located in the top surface of the platform 35. FIG. 3 further illustrates aspheric lenses 130, 110 and 120 which are positioned to focus light entering and exciting the optical unit.

Also shown is the shaft 410 carrying the rotatable platform and connected to the stepper motor for actuating the rotatable platform. Movement of the rotatable platform is not only used to move magnetically attractable matter at the fluid contact surface 57 of the substrate via the magnet 30 but also to switch positions between different optical units and thus allow to direct light of different wavelength to the fluid droplet arranged directly above the opening of the optical unit. In FIG. 3 a further optical unit is shown at the right side of the image. In particular a further emission filter 321 and lens 131 of another optical unit can be seen. In FIG. 3 the temperature control modules (e.g. 53, 52) form an integral part of the substrate and are not covered with a glass slid as illustrated in FIG. 2.

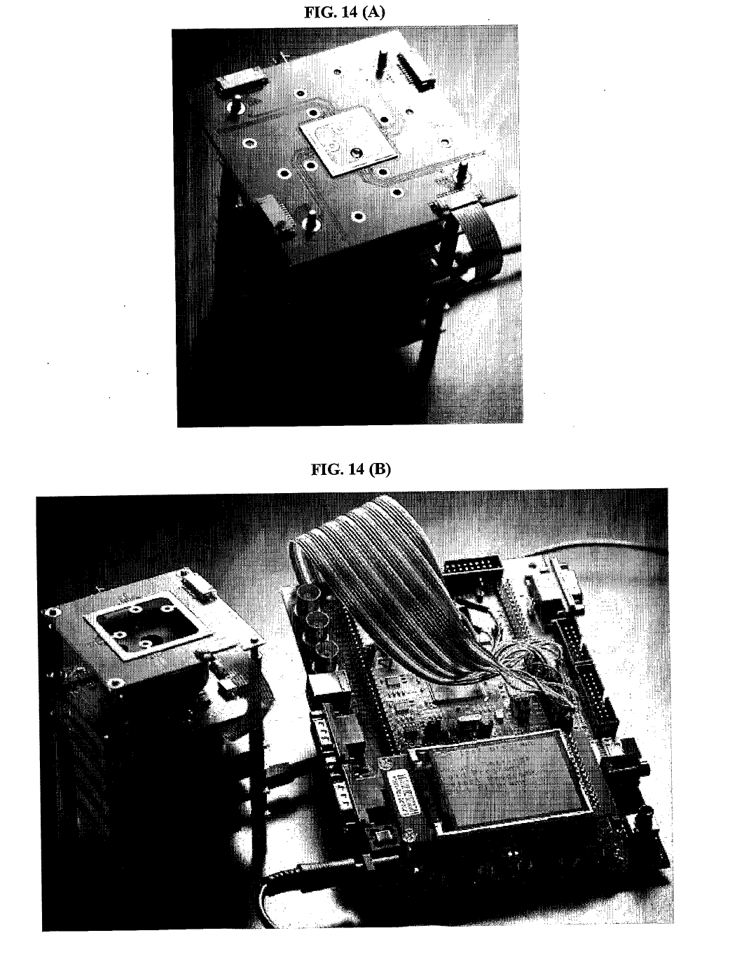

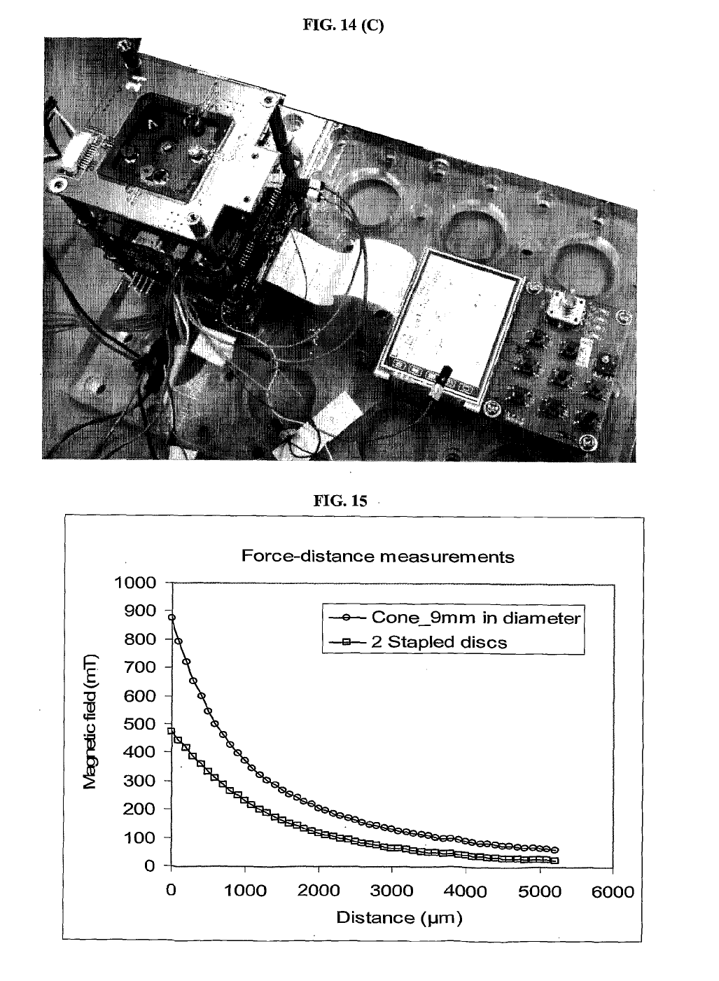

The apparatus can further describe a user interface, e.g. for adapting the apparatus to decentralized point-of-care (POC) tests. Instead of using a combination of PC, oscilloscope and LAB View software, which can also be used, for a microcontroller (MCU) packaged for example with a touch-sensitive color TFT-display. Furthermore, the implementation of a battery (re)charging circuit suited for various battery types enables the use of the apparatus for field-testing (see for example FIG. 14). In an integrated device such as shown in FIG. 14(A) all functional printed circuit board (PCB) modules (power supply, stepper motor, thermal management including temperature control modules, optical detection including optical units and photo detector, and the substrate for carrying out the biological and/or chemical reactions) except microcontroller unit and TFT display were stacked and can easily be replaced swapped for new upgrades. FIGS. 14(B) and (C) show an embodiment together with a microcontroller unit and TFT display. A computer program, for example one that is written in C, controls all essential functions of the apparatus and the user can choose between pre-programmed routine programs for standard chemical and/or biological processes or can create individual sample preparation and/or thermocycling protocols. In another embodiment, the microcontroller and the user interface (e.g. TFT display) are integrated in one single apparatus, for example in form of a tower.

The present invention is further directed to a system comprising an apparatus described herein and magnetically attractable matter described herein.

The inventions illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims and non- limiting examples. In addition, where features or aspects of the invention are described in terms of Markush groups, those skilled in the art will recognize that the invention is also thereby described in terms of any individual member or subgroup of members of the Markush group.

### Experimental Section

A qPCR was carried out with the apparatus of the present invention in presence of a positive internal control. The possibility to process multiple samples allows to examine the actual sample as well as further controls, such as an internal positive control to rule out false-negative results which can be caused for example by a defective apparatus.

System architecture. The power supply with recharging circuit for a lithium ion battery, a microcontroller unit (MCU), touch-sensitive thin film transducer (TFT) display, thermal management, microfluidics, optical detection (Novak, L., Neuzil, P., et al., 2007, Lap Chip, vol. 7, pp. 27), and PCR-chip carrier (Pipper, J., Zhang, Y., et al., 2008, Chem. Int. Ed., vol. 47, pp. 3900) are described in detail elsewhere.

Disposable substrate for fluid contact surface. (Heptadecafluoro-1,1,2,2-tetrahydrodecyl)trimethoxysilane (Gelest)-modified 250 μm-thick D 263 T-glass sheets (Pipper, J., Zhang, Y., et al., 2008, supra) (Schott) were spincoated with a 1% solution of Teflon AF 1600 (DuPont) in FC-40 Fluorinert (3M). The teflonized surfaces had static contact angles with water (Millipore) and M5904-mineral oil (Sigma-Aldrich) of 115±2 and 85±2°, respectively.

Miniaturized optical detection system. The individual parts of the inverted miniaturized optical detection system were manufactured in black anodized aluminum by a combination of vertical milling and electrical discharge machining (SpeedTools). The filter wheel accommodated a permanent neodymium iron boron magnet cone (Neotexx) together with four filter sets (all from Chroma) (Table 1). Possible filters that can be used from Chroma's ET series: #49002 for blue, #49004 for green, #49008 for orange and #49006 for red. In another embodiment, dualband fluorescence filters were used.

TABLE 1
Filter sets used in optical units and fluorescence probes.

| | | | Excitation | Emission | LOD |
|---|---|---|---|---|---|
| Channel | Color | Probe^{a} | (nm) | (nm) | (M) |
| 1 | Blue | FAM-BHQ1 | 470/40 | 525/50 | 11 |
| 2 | Green | HEX-BHQ1 | 545/25 | 605/70 | 462 |
| 3 | Yellow | TxRed-BHQ2 | 560/40 | 630/75 | 14 |
| 4 | Red | Cy5-BHQ3 | 620/60 | 700/75 | 17 |

^{a}For the LOD measurements probes without quenchers were used.

The above filters can be used together with cold or warm white LED. In one embodiment, a warm white NSPL500DS-LED (Nichia) acted as the sole light source. For focusing and/or collimating light, molded aspheric 352330-glass lenses with wavelength-dependent antireflective coatings were used. The individual filter sets were accessed by rotating the rotatable platform to the light source using a 0.9° NEMA size 17 Super Slim Line-stepper motor (Lin Engineering). Thereby, an integrated CNB1302-reflective photosensor (Panasonic) enabled to control the default positions of the rotating rotatable platform. A BPW21R-silicon PD (Vishay) was used for the fluorescence detection during the last 10 seconds of the combined annealing/extension step. Upon completion of a qPCR run, the raw fluorescence readings were transferred as a *.txt file (from the random access memory of the microcontroller unit via a RS232 interface) to a personal computer for further processing.

Data processing. The data processing was performed in Microsoft Excel employing a VBA script described elsewhere (Larionov, A., Krause, A., et al., 2005, BMC Bioinformatics, vol. 6, no. 62, pp. 1).

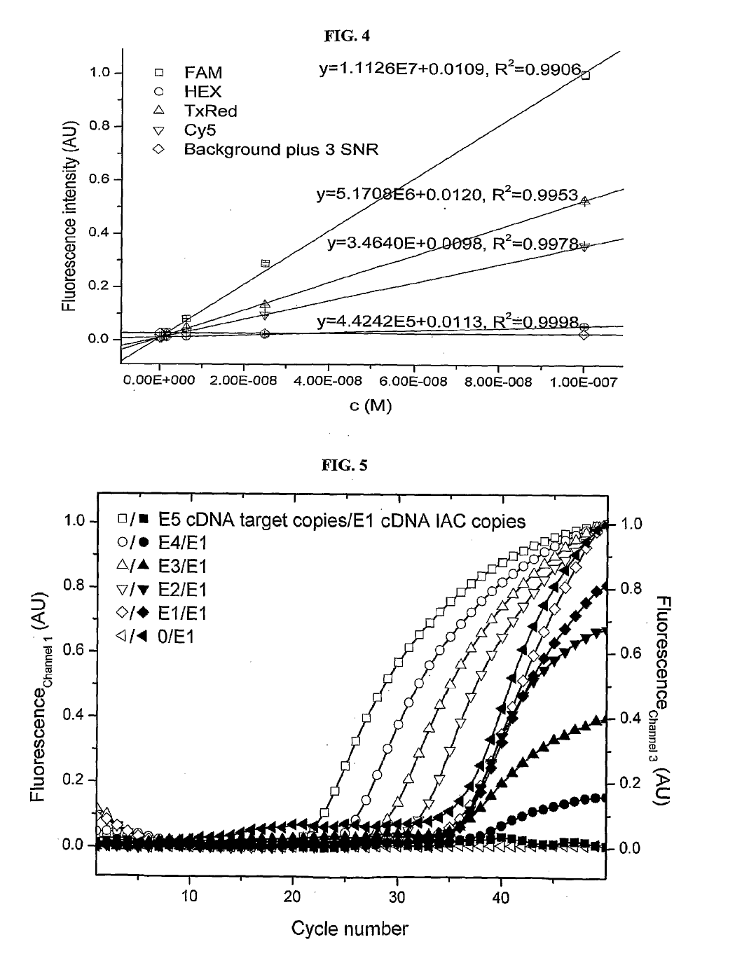

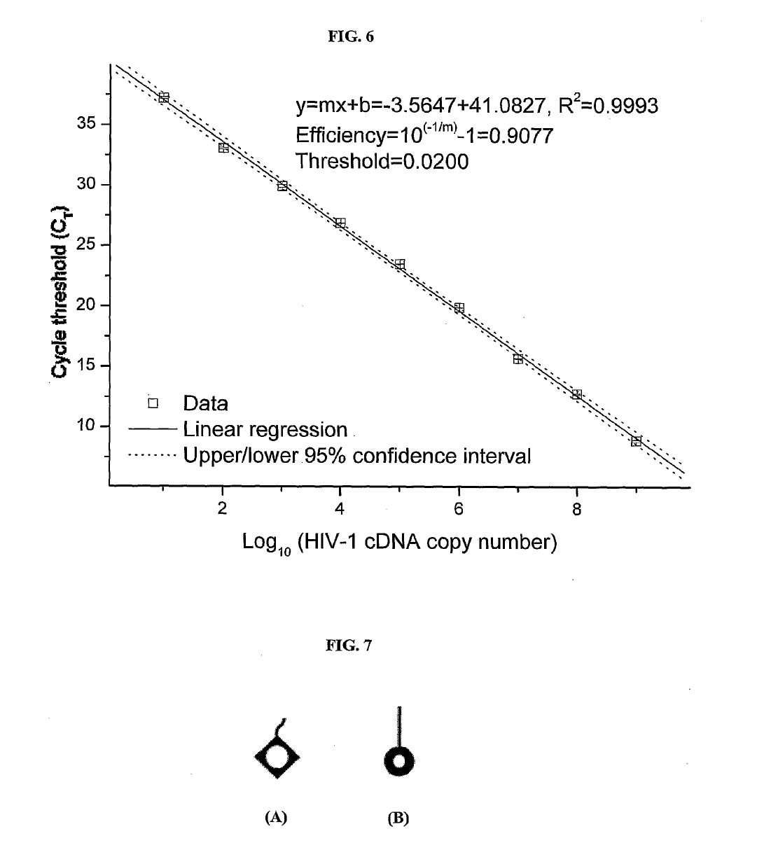

Sensitivity measurements. For assessing the limit of detection (LOD) of the miniaturized optical detection system, the IUPAC definition was followed: c_{LOD}=(3*s_{B})/m, whereby C_{LOD} denotes the concentration of the, analyte at the LOD, s_{B} the standard deviation of the blank measurements, and m the analytical sensitivity, which is expressed as the slope of the calibration curve obtained by linear regression. The probe sequence of the IAC featuring solely different fluorescence donors at its 5′-end was used to generate the calibration plots for each filter set (FIG. 4). Typically, the dilution series' ranged from 10⁻⁷ to 10⁻¹¹M and the diluent was qPCR buffer. All the measurements were recorded at annealing/extension temperature. The LODs measured were: c_{LOD} (FAM)=11 pM, c_{LOD} (HEX)=462 pM, c_{LOD} (TxRed)=14 pM, and c_{LOD} (Cy5)=17 pM.

Sample Isolation. 1000 μL-aliquots of HIV-1 infected blood plasma samples were ultracentrifuged at 24 k g for 1 h at 4° C. Of that, 800 μL of the supernatant were discarded, and the remaining 200 μL containing the viral pellet were extracted using the QIAamp Viral RNA Mini Kit (QIAGEN). Purified RNA was [eluted in 50 μL of TE buffer and] stored at −80° C.

Primer Set and TaqMan Probes Design. The design of the primer set and TaqMan probes targeting the gag gene of HIV-1 were based on alignment data referenced from the HIV Sequence Compendium 2008 (HIV Sequence Compendium 2008, Published by Theoretical Biology and Biophysics, Los Alamos National Laboratory, USA). A competitive qPCR format was chosen, in which both target and IAC made use of one common primer set. The IAC had no sequence homology to HIV-1 and was designed from random synthetic DNA by a polymerase chain assembly, followed by in-vitro transcription. 5′-CTAGCAGTGGCGCCCGAACAG-3′ (SEQ ID NO: 1) and 5′-CCATCTCTCTCCTTCTAGCCTCCGCTAGTCA-3′ (SEQ ID NO: 2) as forward and reverse primers were used, respectively. The TaqMan probes were (FAM)5′-TCTCTCGACGCAGGACTCGGCTTGCTG-3′(BHQ1) (SEQ ID NO: 3) for the target and (TxRed)5′-AGGTCGGGTGGGCGGGTCGTTA-3′(BHQ2) (SEQ ID NO: 4) for the IAC.

qPCR. The qPCR mixture was based on the EXPRESS One-Step SuperScript qRT-PCR Kit (Invitrogen) and composed of 25 μL EXPRESS SuperScript qPCR SuperMix Universal, 10 μL water, 5 μL 10% BSA, 1 μL 10 μM primers each, 0.5 μL 10 μM TaqMan probes each, 1 μl 10 copies μL⁻¹ IAC, 1 μL template cDNA, and 5 μL EXPRESS SuperScript Mix for One-Step qPCR. 1 ng μL⁻¹ tRNA served as negative template control (NTC). Of that, 2.5 μL were used for the miniaturized qPCR and 25 μL for the tube-based PCR using a Mx3000P thermocycler (Stratagene). The thermocycling program comprised a hot start activation at 95° C. for 120 s, followed by 50 thermocycles of denaturation at 95° C. for 10 s, and annealing/extension at 65° C. for 40 s. To counter evaporation, the qPCR mixture was sealed with 10.0 μL mineral oil (Sigma-Aldrich). Finally, the PCR product specificity and yield by capillary electrophoresis (CE) using a Bioanalyzer 2100 (Agilent) was verified.

With reference to FIG. 2 a duplex PCR was carried out. 2.5 μl of the PCR sample overlaid with 12.5 μl of mineral oil were placed on the 9 o'clock position of the fluid contact surface of the device shown in FIG. 2. For the fluorescence readings the measurements were carried out with the blue channel (2 s for the fluorescence reading), and then the rotatable platform was rotated (2×72°=144° which took about 1 s). Thus, the sample was placed before the orange channel (2 s for the fluorescence reading). The rotatable platform was moved until the sample was back in position above the blue channel to wait for the next reading. The rotatable platform was moved back and forth to repeat the readings.

In another example, the sample (together with a suited lysis buffer) was positioned in the 12 o'clock position on the fluid contact surface of the device illustrated in FIG. 2. Further droplets containing the washing solution were placed along the perimeter between the 12 o'clock and 7 o'clock position, and the PCR droplet at the 9 o'clock position. Then, the immobilized RNA/DNA gets dragged through all the droplets along the way (and gets purified) as already described above and finally, the purified RNA/DNA is desorbed into the PCR droplet, in which a qPCR takes place. By placing another small disc magnet under the rotating unit at 9 o'clock, the cone-shaped magnet gets attracted downwards (2 to 3 mm) and looses its ‘contact’ to the magnetic particles due to the increasing distance between magnet and particles located in the droplet at the fluid contact surface (please see FIG. 15 with the force-distance measurements). Thereby, the magnetic attraction between the cone-shaped magnet and the magnetic particles is interrupted between at 9 o'clock, and it is possible to prevent the magnetic particles to ‘rotate’ any further during the fluorescence detection step. In this arrangement, the PCR is taking place in the presence of the magnetic particles (they just stay behind in the PCR droplet). Alternatively, the small magnet can be place anywhere between the 9 and 12 o'clock position, in which case the PCR is carried out in the absence of the magnetic particles. Another possibility would be to create an obstacle (well, hole or slit) between 9 and 12 o'clock, which also would prevent the magnetic particles to rotate further.

1. An apparatus for processing at least one biological and/or chemical sample, the apparatus comprising:
a substrate having a fluid contact surface; wherein the fluid contact surface comprises a texture and a wettability adapted to allow a fluid droplet arrangeable thereon to remain intact upon being contacted with the fluid contact surface of the substrate;
at least one temperature control module arranged to control the temperature in at least one temperature zone at the fluid contact surface of the substrate;
a rotatable platform arranged on the opposite side of the fluid contact surface of the substrate; wherein the rotatable platform comprises:
a magnetic field generator; wherein the magnetic field generator is vertically movable parallel to the rotational axis of the rotatable platform; and
at least one optical unit adapted to emit light of at least one specific wavelength and to detect light of at least one specific wavelength.

2. The apparatus of claim 1, wherein the at least one temperature control module is arranged between the rotatable platform and the fluid contact surface of the substrate.

3. The apparatus of claim 1, wherein the at least one temperature control module forms an integral part of the substrate.

4. The apparatus of claim 1, wherein the magnetic field generator and the at least one optical unit are arranged on a circular orbit on the rotatable platform; wherein the circular orbit is aligned with the at least one temperature zone at the fluid contact surface of the substrate.

5. The apparatus of claim 1, wherein the at least one optical unit comprises:
an excitation light source adapted to provide excitation light;
a detection module adapted to direct light of at least one specific wavelength emitted by a composition comprised in a fluid droplet arrangeable on the fluid contact surface of the substrate to a photo detector;
a light selecting and guiding device; wherein the light selecting and guiding device is positioned
a) to direct the excitation light toward the fluid droplet arrangeable on the fluid contact surface of the substrate; and
b) to direct light inclining from the fluid droplet arrangeable on the fluid contact surface of the substrate to the detection module.

6. The apparatus of claim 5, wherein the excitation light source is an excitation filter adapted to filter light of at least one specific wavelength received from a light emitting system.

7. The apparatus of claim 5, wherein the detection module comprises an emission filter adapted to filter light of at least one specific wavelength emitted by a composition comprised in a fluid droplet arrangeable on the fluid contact surface of the substrate.

8. (canceled)

9. The apparatus of claim 5, further comprising a lens positioned on the side of the detection module which is opposite the side on which the light selecting and guiding device is positioned to focus incident light directed to the detection module.

10. The apparatus of claim 5, further comprising a lens positioned to focus excitation light received from the light selecting and guiding device onto the fluid droplet.

11. (canceled)

12. The apparatus of claim 1, further comprises comprising a support structure supporting the substrate, the at least one temperature control module and the rotatable platform.

13. The apparatus of claim 12 wherein the support structure comprises at least one photo detector positioned to detect inclining light from a fluid droplet arrangeable at the fluid contact surface of the substrate and passed through the optical unit.

14. The apparatus of claim 12, wherein the support structure comprises at least one light emitting system positioned to couple light into the at least one optical unit.

15. The apparatus of claim 14, wherein the at least one light emitting system further comprises a lens for directing light onto the optical unit.

16. (canceled)

17. The apparatus of claim 1, wherein the apparatus comprises at least two or at least three or at least four temperature control modules.

18. The apparatus of claim 1, wherein the movement of the rotatable platform is controlled by a stepper motor.

19. The apparatus of claim 1, wherein the at least one temperature control module comprises:
a heater;
a heat conductor; and
a temperature sensor;
wherein the temperature sensor is adapted to detect and control the temperature in the at least one temperature zone at the fluid contact surface of the substrate via the heat conductor.

20. The apparatus of claim 19, wherein the heater and the sensor are concentric.

21.-22. (canceled)

23. The apparatus of claim 1, wherein the apparatus comprises at least two temperature zones and wherein the at least two temperature zones are thermally isolated from each other.

24.-28. (canceled)

29. A system comprising:
an apparatus for processing at least one biological and/or chemical sample, the apparatus comprising:
a substrate having a fluid contact surface; wherein the fluid contact surface comprises a texture and a wettability adapted to allow a fluid droplet arrangeable thereon to remain intact upon being contacted with the fluid contact surface of the substrate:
at least one temperature control module arranged to control the temperature in at least one temperature zone at the fluid contact surface of the substrate;
a rotatable platform arranged on the opposite side of the fluid contact surface of the substrate; wherein the rotatable platform comprises:
a magnetic field generator; wherein the magnetic field generator is vertically movable parallel to the rotational axis of the rotatable platform; and
at least one optical unit adapted to emit light of at least one specific wavelength and to detect light of at least one specific wavelength; and
magnetically attractable matter.

30.-31. (canceled)

32. A method of processing at least one biological and/or chemical sample, wherein the method comprises:
disposing at least one fluid droplet onto a fluid contact surface of a substrate of the apparatus for processing at least one biological and/or chemical sample, the apparatus comprising:
a substrate having a fluid contact surface; wherein the fluid contact surface comprises a texture and a wettability adapted to allow a fluid droplet arrangeable thereon to remain intact upon being contacted with the fluid contact surface of the substrate;
at least one temperature control module arranged to control the temperature in at least one temperature zone at the fluid contact surface of the substrate;
a rotatable platform arranged on the opposite side of the fluid contact surface of the substrate; wherein the rotatable platform comprises:
a magnetic field generator; wherein the magnetic field generator is vertically movable parallel to the rotational axis of the rotatable platform; and
at least one optical unit adapted to emit light of at least one specific wavelength and to detect light of at least one specific wavelength; and
performing a process on the biological and/or chemical sample in the at least one fluid droplet;
wherein the fluid droplet comprises an inner phase and an outer phase,
wherein the outer phase is immiscible with the inner phase, and the outer phase is surrounding the inner phase, and
wherein the inner phase comprises the biological and/or chemical sample, and the inner phase is shielded from the environment by the outer phase;
wherein the fluid droplet comprises magnetically attractable material.

33.-38. (canceled)


SEQUENCE LISTING (4 sequences)

SEQ ID NO: 1 - DNA, 21 bp, Artificial
  Forward Primer
ctagcagtggcgcccgaacag

SEQ ID NO: 2 - DNA, 31 bp, Artificial
  Reverse Primer
ccatctctctccttctagcctccgctagtca

SEQ ID NO: 3 - DNA, 27 bp, Artificial
  TaqMan Probe for the target
tctctcgacgcaggactcggcttgctg

SEQ ID NO: 4 - DNA, 22 bp, Artificial
  TaqMan probe for the IAC
aggtcgggtgggcgggtcgtta